(19) United States
(12) Patent Application Publication
(10) Pub. No.: US 2023/0075953 A1
(43) Pub. Date: Mar. 9, 2023

(54) GADUSOL-CONTAINING SUNSCREEN FORMULATIONS

(71) Applicant: Arcaea, LLC, Boston, MA (US)

(72) Inventors: Khaled H. Almabruk, Corvallis, OR (US); Alan T. Bakalinsky, Corvallis, OR (US); Atchara Sripeng, Corvallis, OR (US); Nathan Alexander, Corvallils, OR (US); Jennifer Cookson, Arlington, MA (US); Margaret Sullivan, Medfield, MA (US); Kimberly Biedermann, Quincy, MA (US); Irwin Palefsky, Elwood, NY (US); Kathryn Pettinger, Corvallis, OR (US)

(21) Appl. No.: 17/823,939

(22) Filed: Aug. 31, 2022

Related U.S. Application Data
(63) Continuation-in-part of application No. PCT/US22/18377, filed on Mar. 1, 2022.
(60) Provisional application No. 63/155,104, filed on Mar. 1, 2021.

(51) Int. Cl. A61K 8/34; A61K 8/27; A61K 8/44; A61Q 17/04

(52) U.S. Cl. CPC A61K 8/347; A61K 8/27; A61K 8/44; A61Q 17/04; A61K 2800/30; A61K 2800/412; A61K 2800/413

(57) ABSTRACT

Embodiments of sunscreen formulations are disclosed herein. In one example, a sunscreen formulation includes a) a physical/inorganic sunscreen active ingredient at from about 5% to about 25% on a weight/weight (w/w) basis, wherein the active ingredient generates a first sun protection factor (“SPF”) value to a sunscreen formulation; b) gadusol; and c) a plurality of ingredients configured to deliver ingredients a) and b) in the sunscreen formulation, wherein the gadusol is present in the sunscreen formulation in an amount sufficient to generate a SPF booster effect in the sunscreen formulation.

### CROSS-REFERENCE TO RELATED APPLICATIONS

This application is a Continuation-in-Part application of PCT Application No. PCT/US22/18377, filed Mar. 1, 2022, which claims priority to Provisional Patent Application Ser. No. 63/155,104, filed Mar. 1, 2021. The contents of each of these applications are each herein incorporated by reference herein in their entireties.

### BACKGROUND

It is well known that UV radiation (that is, wavelengths ranging from about 290 nm to 400 nm) can cause damage to the human epidermis. More particularly, UV rays with wavelengths ranging from 320 to 400 nm (known as UVA) may cause hyperpigmentation of the skin and long-term damage therein (such as wrinkles and fine lines), and rays with wavelength ranging from 280 to 320 nm (known as UVB) can cause erythema/sunburn. In order to prevent or minimize damage or harmful effects caused by UV radiation, skin care compositions containing UV-blocking compounds in varying concentrations (also called sunscreen compositions or formulations) are recommended for use.

Sunscreen products are regulated as over-the-counter drugs by the US Food and Drug Administration (“FDA”), and as cosmetic products in the EU, Japan, Korea, and other countries. These formulations are topically applied products indicated to help prevent sunburn, which is an acute effect of over-exposure to sun. As would be understood, the amount of sun needed to impart “over exposure” will differ according to a person's specific skin type. While sunscreen products have been used by many consumers to prevent sunburn for many years, more recently sunscreen products have included broad-spectrum protection, that is, both UVB and UVA blocking activity. To this end, many sunscreen products today are also formulated to reduce the risk of skin cancer and early skin aging as caused by long-term exposure to UVA wavelengths. Broad spectrum sunscreens protect from UVB radiation (about 290 to 320 nm), which causes skin cancer and sunburn, as well as UVA radiation (320 to 400 nm), which contributes to skin cancer and causes aging and wrinkling of skin.

By way of explanation, sunscreens work by preventing and minimizing the damaging effects of the ultraviolet sun rays following exposure to the sun, specifically by preventing two of the sun's three types of UV rays—UVA and UVB—from penetrating the wearer's skin. Ultraviolet filters, also referred to as “sunscreen actives,” are the ingredients present in formulations that interfere directly with the effects of the sun on skin via absorption, reflection or dispersion of solar energy. Currently, the most common sunscreen actives in use in the US are UV filters avobenzone, homosalate, octinoxate, octocrylene, octisalate, oxybenzone, titanium dioxide, and zinc oxide.

Chemical sunscreens absorb UV light and convert it into heat energy that is then released from the skin. In conventional formulations, organic sunscreens generally provide cosmetically acceptable aesthetics upon application. A much larger number of chemical sunscreen actives are in use in the European Union (“EU”). Most chemical filters protect against UVB rays, and few of them also protect partially from UVA rays.

While proven over the years to provide good protection from well-formulated sunscreen products, in recent years, there have been increasing concerns associated with the use of chemical sunscreens. For example, oxybenzone has been used in American sunscreen for decades. However, it has also been found in breast milk, urine, and amniotic fluid. Some animal studies have also demonstrated that oxybenzone exhibits estrogenic and antiandrogenic activity in laboratory animals and therefore, may pose a risk for potential endocrine disorders from long term use. In 2019, the FDA determined that none of the currently approved chemical sunscreens in use in the U.S. can be considered to be “Generally Recognized as Safe and Effective” (“GRASE”) for use.

Beyond concerns about negative biological effects on the humans who use them, chemical sunscreens have been associated with environmental contamination, with oxybenzone, octocrylene, octinoxate being identified in fresh and saltwater. Based on their physical and chemical properties, some UV filters have the potential to persist and accumulate in aquatic ecosystems. Bioaccumulation refers to the accumulation of a chemical into an organism via all routes of exposure. UV filters exhibit a range of bioaccumulation potentials, driven primarily by the lipophilicity of the compound and the metabolism of the parent compound by biota. High-quality, laboratory-based bioaccumulation or bioconcentration factors available for avobenzone, octocrylene, octinoxate, oxybenzone, and homosalate reveal a low to moderate bioaccumulation in a number of marine animals/organisms.

As would be appreciated, a common use case—if not the most common use case—for sunscreen products is for sun protection in and around water. One study posits that about 25% of the sunscreen worn by a person who swims is released into the water. Researchers have estimated that 14,000 tons of sunscreen are washed into the ocean each year. The sunscreen that goes into the ocean does not spread out across the entire planet. Instead, it accumulates where people are diving and swimming the most—areas like coral reefs, kelp forests, and marine reserves that host significant amounts of marine life. Today, much focus is on coral reefs, where some research indicates that wide-spread use of chemical/organic-containing sunscreen product contributes to coral bleaching. While there may currently be inconsistent in vivo data about actual negative effects on marine life, a number of bans have been enacted on chemical sunscreen ingredients (e.g., oxybenzone, octinoxate, octocrylene, homosalate, 4-methylbenzylidene camphor, para-aminobenzoic acid, parabens, and triclosan) to prophylactically protect coral reefs.

Physical/inorganic sunscreens actives act by absorbing, reflecting and scattering the UV light thereby protecting the skin. ZnO and TiO₂ have been used as particulate sunscreen ingredients for many years and are, in fact, the only sunscreens currently considered by the FDA to be GRASE when used correctly. However, the reflective nature of inorganic sunscreen materials can also leave a user with excessive shine and/or a white/blue residue on their skin, which can limit their cosmetic acceptance. Of course, to be effective to reduce or prevent sunburn and sun damage, a sunscreen has to actually be worn by a person. A cosmetically unacceptable sunscreen will often not be worn as needed, which means that although they are GRASE materials and effective as sunscreen active ingredients, many persons who need sun protection may forgo use of sunscreen products containing either or both of ZnO and TiO₂ if doing so leaves them with an unacceptable appearance. In other words, while ZnO and TiO₂ may be GRASE according to applicable regulatory agencies, they cannot provide an effective form of sun protection for those specific users who find their use cosmetically unacceptable.

To counter the problem of skin whitening, nanoparticle forms of ZnO and TiO₂ (average particle size of <100 nm) have been formulated into many sunscreen products. With the use of ZnO and TiO₂ nanoparticles in well-formulated sunscreens, undesirable shine and whiteness can be reduced or even eliminated (on some skin tones) when the sunscreen is applied by a user. However, in some formulations, the effectiveness of these nanoparticle materials as sunscreen active ingredients can also be reduced. To this end, the efficacy of ZnO and TiO₂ as sunscreen actives can be related to the degree of coverage of these reflective particles on the skin of a user; if there is not enough ZnO or TiO₂ on the skin surface after application of a physical/inorganic sunscreen active formulation, the intended amount of sun protection may not be generated. This might be found when a nanoparticle-containing sunscreen formulation is applied to a skin surface in the first order at least because smaller particles may generate a different reflectivity than larger particles. Also, some research indicates that nanoparticles may enter the stratum corneum instead of remaining on the outer surface of the skin. This can alter specific nanoparticles attenuation properties due to particle-particle, particle-skin, and skin-particle-light physicochemical interactions. It may then be contended that nanoparticle-containing sunscreen products may exhibit a lesser degree of effectiveness than those containing larger inorganic particles.

In addition to surface coverage difficulties that may exist in some formulations, some consumers may wish to avoid nanoparticles. While the various governmental regulatory agencies throughout the world have found that these sunscreen active ingredients to be GRASE, several studies have identified ZnO and TiO₂ nanocomposites in the urine, blood and feces of persons who have applied sunscreen products. This indicates that there may be at least some penetration into the skin upon application. Although the current research provides conflicting information about the extent of such penetration and the negative effects that might result, it might be contended that some consumers may be concerned that inorganic nanoparticles are not safe enough for them. As such, these consumers may seek to avoid sunscreen products that contain nanoparticles if only because the risk of using these products is currently unsettled.

Substitution of chemical sunscreens with ZnO and TiO₂ do not necessarily make these products fully safe for marine life. To the contrary, some recent research has shown that nanocomposite inorganic UV filters might exhibit bioaccumulation in aquatic environments. As a result, there is an emerging concern that use of ZnO and TiO₂ nanoparticles in sunscreen products may cause toxic effects on marine life. While research on the use of ZnO and TiO₂ in nanoparticle form as sunscreens is still being conducted, it might be argued that the long-term health and environmental effects of sunscreens comprising nanoparticle-sized inorganic sunscreens remain uncertain. Thus, there currently is a question of whether sunscreens made using ZnO and TiO₂ nanoparticles are either safe or both safe and effective.

In view of the above, there is a need for improvements in sunscreen products. Specifically, there is a need for sunscreen products that are both safe for users and the environment and effective in providing sun protection to users.

### SUMMARY

In one embodiment, a sunscreen formulation includes a) a physical/inorganic sunscreen active ingredient at from about 5% to about 25% on a weight/weight (w/w) basis, wherein the active ingredient generates a first sun protection factor (“SPF”) value to a sunscreen formulation; b) gadusol; and c) a plurality of ingredients configured to deliver ingredients a) and b) in the sunscreen formulation, wherein the gadusol is present in the sunscreen formulation in an amount sufficient to generate a SPF booster effect in the sunscreen formulation.

It is to be understood that the summary above is provided to introduce a simplified selection of concepts that are described further in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a chemical structure of gadusol.

FIGS. 2A-2C show the chemical structures for gadusporines A, B, and C, respectively.

FIG. 3 shows an example UV spectrum for gadusol and gadusporine A.

FIG. 4 shows an example UV spectrum for gadusol and gadusporine A across UVA through UVC.

FIG. 5 shows the effect of gadusol addition on in vivo SPF activity of 10% ZnO-containing sunscreen formulations.

FIG. 6 shows the in vitro absorbance from 290-400 nm of 0.5% gadusol/10% ZnO-containing formulation and a 10% ZnO-containing formulation with no added gadusol.

FIG. 7 shows the in vitro absorbance from 290-400 nm of a gadusol-containing sunscreen formulation.

FIG. 8 shows the in vitro whitening effects of various ZnO-containing sunscreen formulations with and without gadusol.

FIG. 9 is a photograph of various ZnO-containing sunscreen formulations with and without gadusol.

FIG. 10 is a photograph and spectrophotometric data of the whitening effects on a Type1 Fitzpatrick Phototype of various ZnO-containing sunscreen formulations with and without gadusol.

FIG. 11 is a photograph and spectrophotometric data of the whitening effects on a Type 4 Fitzpatrick Phototype of various ZnO-containing sunscreen formulations with and without gadusol.

FIG. 12 is a photograph and spectrophotometric data of the whitening effects on a Type 5 Fitzpatrick Phototype of various ZnO-containing sunscreen formulations with and without gadusol.

### DETAILED DESCRIPTION

Many aspects of the disclosure can be better understood with reference to the Figures presented herewith. The Figures are intended to illustrate the various features of the present disclosure. While several implementations may be described in connection with the included drawings, there is no intent to limit the disclosure to the implementations disclosed herein. To the contrary, the intent is to cover all alternatives, modifications, and equivalents.

Before the present disclosure is described in greater detail, it is to be understood that this disclosure is not limited to particular aspects described, and as such may, of course, vary. It is also to be understood that the terminology used herein is for the purpose of describing particular aspects only and is not intended to be limiting. The skilled artisan will recognize many variants and adaptations of the aspects described herein. These variants and adaptations are intended to be included in the teachings of this disclosure and to be encompassed by the claims herein.

All publications and patents cited in this specification are herein incorporated by reference as if each individual publications or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publications by virtue of prior disclosure. Further, the publication dates provided could be different from the actual publication dates that may need to be independently confirmed.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Although any methods and materials similar or equivalent to those described herein can also be used in the practice or testing of the present disclosure, the various methods and materials suitable for use with the various inventions disclosed herein are now described. Functions or constructions well-known in the art may not be described in detail for brevity and/or clarity.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”

As used in the specification and the appended claims, the singular forms “a,” “an” and “the” include plural referents unless the context clearly dictates otherwise. By way of example, the term “gadusporine” can refer to one of gadusporine A, gadusporine B, or gadusporine, or collection of two or three of the gadusporines A, B, and C.

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±20%, ±15%, ±10%, ±9%, ±8%, ±7%, ±6%, or ±5% of the specified value, e.g., about 1″ refers to the range of 0.8″ to 1.2″, 0.8″ to 1.15″, 0.9″ to 1.1″, 0.91″ to 1.09″, 0.92″ to 1.08″, 0.93″ to 1.07″, 0.94″ to 1.06″, or 0.95″ to 1.05″, unless otherwise indicated or inferred. It is understood that where “about,” “approximate,” or “at or about” is used before a quantitative value, the parameter also includes the specific quantitative value itself, unless specifically stated otherwise.

Any ratios, concentrations, amounts, and other numerical data can be expressed herein in a range format. Such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘x, y, z, or greater’ should be interpreted to include the specific ranges of ‘about x’, ‘about y’, and ‘about z’ as well as the ranges of ‘greater than x’, greater than y′, and ‘greater than z’. In some aspects, the term “about” can include traditional rounding according to significant figures of the numerical value. In addition, the phrase “about ‘x’ to ‘y’” includes “about ‘x’ to about ‘y’”.

The term “substantially” is meant to permit deviations from the descriptive term that do not negatively impact the intended purpose. All descriptive terms used herein are implicitly understood to be modified by the word “substantially,” even if the descriptive term is not explicitly modified by the word “substantially.”

“SPF” stands for Sun Protection Factor. In the U.S., sunscreens are regulated by the Food and Drug Administration (“FDA”) to ensure they meet safety and effectiveness standards. In Europe and in some other countries/jurisdictions, sunscreens are regulated as cosmetics, not as drugs, and are subject to different marketing requirements. Any sunscreen sold in the U.S. is regulated as a drug because it makes a drug claim which is indicated to help prevent sunburn or to decrease the risks of skin cancer and early skin aging caused by the sun.

As defined by the U.S. FDA, SPF is a measure of how much solar energy (UV radiation) is required to produce sunburn on protected skin (i.e., in the presence of sunscreen) relative to the amount of solar energy required to produce sunburn on unprotected skin. Because of the various factors that impact the effects that an amount of solar radiation may have on a specific person, SPF does not reflect the time a user can safely remain in the sun. That is, SPF does not inform consumers about the time that can be spent in the sun without experiencing acute sun damage, that is, getting a “sunburn.” Rather, SPF is a relative measure of the amount of sunburn protection provided by a sunscreen having a particular SPF value. It allows consumers to compare the level of sunburn protection provided by different sunscreens. Consumers understand that SPF 30 sunscreens provide more sunburn protection than SPF 8 sunscreens. Each consumer will exhibit her own propensity to acquire a sunburn during a period of time spent in the sun depending on her characteristics. For example, people with fairer complexions will typically attain a sunburn more quickly—and in a more intense form—than people with darker complexions. Age, medicine intake, illness, and other characteristics can also be relevant to someone's reactivity UV rays when in the sun. Irrespective of one's complexion or other unique and personal characteristics, the amount of sunburn likely to be generated during midday in the summer months will be greater than the amount of sunburn likely earlier or later in the same day, which means a higher or lower SPF sunscreen can be indicated for different times of the day.

SPF measures the amount of UV radiation exposure it takes to cause sunburn when using a sunscreen compared to how much UV exposure it takes to cause a sunburn when not using a sunscreen. The product is then labeled with the appropriate SPF value. Because SPF values are determined from testing that measures protection against sunburn—that is, the acute damage—caused by UVB radiation. In the absence of a “broad-spectrum protection” claim, SPF values only indicate a sunscreen's UVB protection. In other words, the SPF value indicates the level of sunburn protection provided by the sunscreen product.

All products sold as “sunscreens” in the U.S. must be tested according to an SPF test procedure. The SPF value indicates the level of sunburn protection provided by the sunscreen product. In some contexts herein, SPF is associated with a value generated from an in vivo test method. In other contexts, SPF is associated with an in vitro method. There can be some differences in an SPF that is generated using either method, at least because the in vivo method utilizes actual persons who will exhibit variability in response. The in vitro method utilizes a standardized simulated skin substrate, which can be more consistent from sample to sample. Each SPF testing method is described herein.

SPF can be measured in vivo according to the methodology set out in FDA 2011 Final Rule issued on Jun. 17, 2011, by the Federal Register. This method is very similar to the ISO 24444:2010 method used to measure SPF in the EU, as well as other countries. In fact, both methods are based on the International Sun Protection Factor (SPF) test method, COLIPA: Bruxelles, Belgium, 2006. The slight differences between the ISO and FDA methods are understood to generate no meaningful difference in the results. Therefore, the SPF values obtained with the two methods are comparable. The main differences between the methods are the following:—the calibration times of the UV source (each 12 months for the FDA, each 18 months for the ISO);—the reference sunscreen products (P2 for the FDA and P2, P3 or P7 for the ISO); —progressions of exposure times used. The disclosures of each of these in vivo SPF testing methods are incorporated herein their entirety by this reference.

The in vivo test measures the amount of UV radiation exposure it takes to cause sunburn when a person is using a sunscreen in comparison to how much UV exposure it takes to cause a sunburn when they do not use a sunscreen. The product is then labeled with the appropriate SPF value indicating the amount of sunburn protection provided by the product.

SPF can also be evaluated using an in vitro method that utilizes a synthetic/simulated skin as a test substrate. In this regard, VitroSkin® is an advanced testing substrate that effectively mimics the surface properties of human skin. It has been formulated to have topography, pH, critical surface tension, chemical reactivity and ionic strength that is similar to human skin.

Sunscreens that pass a broad-spectrum test will have demonstrated that they also provide UVA protection that is proportional to their UVB protection. To pass the broad-spectrum test, sunscreens with higher SPF values will provide higher levels of UVA protection as well. Therefore, under current U.S. labeling requirements, a higher SPF value for sunscreens labeled “Broad Spectrum SPF [value]” will indicate a higher level of protection from both UVA and UVB radiation. Furthermore, as regards the labeling of the products, the FDA method establishes that the maximum SPF value to be reported on the label is 50+. According to the FDA, “Broad Spectrum” UVA protection can be indicated on the label only for sunscreens having SPF≥15 and a critical wavelength of at least 370 nm. In the EU, broad spectrum protection is determined by the ratio of UVA to UVB protection. A product must achieve a ratio of 1/3 UVA/UVB protection in order to achieve the broad-spectrum label claim.

An amount of UVA protection can also be determined by the UVA-PF test and then calculated based on the SPF of the product. The UVA-PF test is similar to SPF in that it is a measure of how well a sunscreen protects the skin from UVA radiation. It is performed by irradiating the skin (or a plastic slide/sheet) with UVA. This can be done in vivo with human skin (similarly to SPF), or in vitro (using a skin analog), where the test can be performed by applying the sunscreen to an acrylic or plastic (PMMA) slide and measuring how much UVA passes through the slide with or without the sunscreen.

Other methods of calculating UVA protection include PPD, which means “persistent pigment darkening.” PPD is the second phase of “tanning” and is believed to be strongly activated by UVA radiation. The PPD method measures the amount of UVA radiation required to produce the first unambiguous pigmented reaction on protected and unprotected skin in a human subject. The PPD method appears to correlate well with in vitro UVA-PF testing. The results of in in vivo PPD testing can be presented to users as follows:

PA+=PPD of 2-4

PA++=PPD of 4-8

PA+++=PPD of 8-16

PA++++=PPD of 16 or more.

For sunscreen products claiming to be “water resistant,” the SPF is measured after two 20 min immersions (40 min immersion in total) in a spa-pool fitted with a water recirculation device containing water maintained at constant temperature between 27° C. and 31° C. A sunscreen product can claim to be “water resistant” if the SPF value after immersion is equal to more than 50% of the value found before immersion (90% lower unilateral confidence limit for the mean percentage of water resistance retention≥50%). To give an example, a 30 SPF product can claim “water resistant” if it keeps its SPF value higher than 15 after immersion in water.

Sunscreen, also known as “sunblock” or “sun cream,” is a photoprotective topical product for the skin that mainly absorbs, or to a lesser extent reflects, some of the sun's ultraviolet (UV) radiation and thus helps protect against sunburn and, when used consistently and properly over a period of time, reduce the possibility that a person may develop skin cancer. Sunscreen comes in many forms, including: lotions, creams, sticks, gels, oils, butters, pastes, and sprays. A manufacturer provides the consumer with directions for using the products according to the form, as well as various use cases (e.g., whether for children/adults, activities while in the sun, etc.)

Conventionally, sunscreens are classified as having either or both of chemical/organic and physical/inorganic (i.e., zinc oxide and titanium dioxide) active ingredients.

Chemical/organic sunscreens that may be in use in the U.S. today include oxybenzone, avobenzone, cinoxate, homosalate, octinoxate, octisalate, octocrylene, dioxybenzone, ensulizole, meradimate, padimate 0, and sulisobenzone. As of late 2021, the FDA has indicated that use of sunscreens including these ingredients cannot be considered to be GRASE when used as directed. Additional testing is currently underway to generate sufficient data to qualify the continued use of these materials as active ingredients in sunscreen formulations in the U.S. Regardless of whether one or more of these materials are later approved for use after completion of safety testing, as discussed previously, it can be expected that these materials may still be considered at some level to be unsafe for aquatic species when introduced into marine environments, as is inevitable during common usage of sunscreen products. Moreover, some consumers may still seek to avoid chemical/organic sunscreen active materials regardless of whether they are deemed to be GRASE by the FDA in the future.

Sunscreens relying in whole or in part on physical/inorganic sunscreen actives to impart an intended SPF to a consumer in use incorporate either or both of zinc oxide (“ZnO”) and titanium dioxide (“TiO₂”) to reflect, scatter, and/or absorb UV rays. As of 2021, the US FDA considers sunscreens containing ZnO and TiO₂ at up to 25% to be GRASE. Zinc oxide can be a primary physical/inorganic sunscreen active ingredient used in sunscreen formulations because it provides strong sun protection when formulated correctly and worn in accordance with manufacturer's instructions. Notably, ZnO does not break down in the sunlight and offers good protection against UVA rays, also. Titanium dioxide that can be ingested is considered to be a carcinogen and, as such, has been banned for use in foods in the European Union as of late 2021. While the EU still allows nanoparticles of TiO₂ to be used in cosmetics—a category that includes sunscreens as a regulatory class of consumer products—it could be expected that TiO₂ may be subjected to additional regulations in the future. Nanocomposites also may be discouraged in spray sunscreen formulations.

Moreover, while research regarding the penetration of inorganic sunscreen nanoparticles into the stratum corneum and/or possible negative systemic effects to a user are not yet clear, as noted previously, inorganic nanoparticles have been detected in urine, blood, and feces of humans after use of sunscreen products, as well as being taken up by some aquatic life. Thus, some consumers may desire to avoid nanoparticles in their sunscreen formulations in favor of so-called “natural” sunscreen formulations. While there is no regulated category of “natural sunscreen products” in either the US or the EU, an informal marketing category having this name signifies that the subject product uses ZnO or TiO₂ as the active sunscreen ingredient in a “non-nano” form, that is, having an average particle size of greater than 100 nm. It is understood that while information regarding the nano-particle forms of ZnO and TiO₂ might still be developing, the safety of larger physical/inorganic sunscreen active particles—in particular, ZnO—is largely settled for both humans and in the context of marine life that may be subjected to sunscreen active ingredients from user wash off.

While physical/inorganic sunscreen materials are currently recognized as GRASE at up to 25% in both the US and the EU, neither of these materials are typically used at these levels in sunscreen products, especially when particles having an average size of greater than about 100 nm are used in “natural sunscreen” formulations. As noted previously, physical/inorganic sunscreen products, especially those having average particle sizes of greater than about 100 nm can tend to leave a white and/or bluish coatings and create shine on consumers when used at higher levels, especially for consumers with darker skin tones, that is those consumers having Phototypes V and VI on the Fitzpatrick Scale. Even for nanosized ZnO and TiO₂. Whitening and/or bluing on very dark skin can remain problematic for sunscreen formulators when SPF values of 15 or more may be relevant in a sunscreen formulation.

Classification of skin types according to Fitzpatrick Phototype Classification are as follows:

Type 1 always burns, never tans

Type 2 usually burns, tans with difficulty

Type 3 sometimes burns, sometimes tans

Type 4 burns minimally, always tans

Type 5 rarely burns, tans profusely

Type 6, never burns, deeply tans

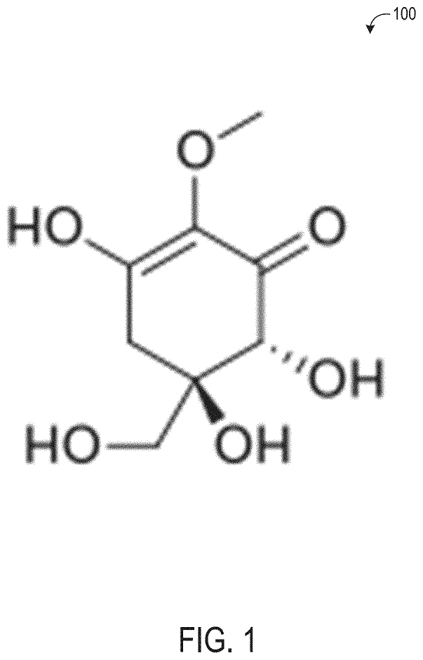

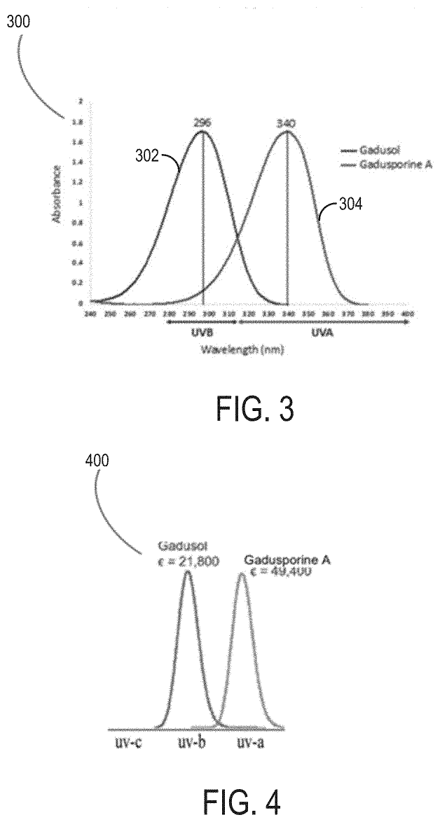

Gadusol is a compound having the structure shown in FIG. 1 as compound 100, and UV absorbance characteristics shown in FIGS. 3 and 4. Gadusol was originally identified in cod roe and has since been discovered in the eyes of the mantis shrimp, sea urchin eggs, sponges, and in the dormant eggs and newly hatched larvae of brine shrimps. Certain fish, such as zebrafish, synthesize gadusol naturally. Gadusol is not found in mammals. The compound is in the class of naturally occurring materials that confer sun protection to the plants and animal species that possess them, with the sun protective ability of gadusol being significant among the various varieties of mycosporine-like amino acids (“MAA's”). Because of this property, gadusol and the related MAA materials can also colloquially be termed “natural sunscreens.” Although naturally occurring in very small amounts in the representative plants and animals, a method to produce this compound biosynthetically has recently been developed, which enhances its usefulness for sunscreen products formulated for humans. Examples of suitable gadusol biosynthesis methodologies are described in U.S. Pat. No. 11,072,806, the disclosure of which is incorporated herein in its entirety by this reference. It is expected that other methodologies may be developed to biosynthesize gadusol in the future, and this disclosure is intended to cover such after-invented biosynthesis pathways.

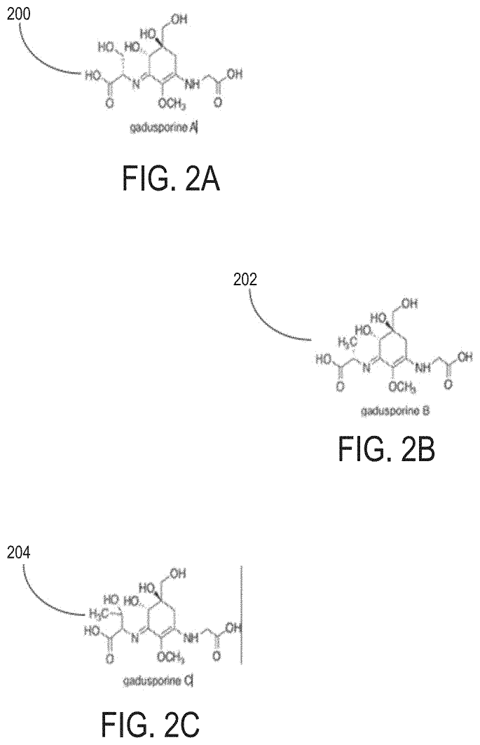

Gadusporines are structurally similar to MAAs. Three gadusporines (A, B, and C) have been reported, among which gadusporine A was found to be the major form (˜90%). The structures of gadusporines A, B, and C are shown in FIGS. 2A, 2B, and 2C as compounds 200, 202, and 204, respectively. The gadusporines are stable water-soluble molecules with excellent UVA-absorption properties as shown in FIGS. 3 and 4. Due to their structural similarities to MAAs, they are also expected to have similar or better antioxidant, wound healing and anti-aging properties. The main difference is that the core cyclic unit of the gadusporines is UVB-absorbing gadusol while in the MAAs, it is 4-deoxygadusol. The biosynthesis of gadusporines is described in US Patent Publication No. US2021/0071184, the disclosure of which is incorporated herein in its entirety by this reference. It is expected that other methodologies may be developed to biosynthesize one or more of the gadusporines in the future, and this disclosure is intended to cover such after-invented biosynthesis pathways.

A “sunscreen booster” is a sunscreen formulation ingredient that may not impart significant inherent UV blocking potential on its own at the concentration used, but when paired with sunscreen active ingredients (e.g., ingredients approved by relevant regulatory agencies) and other non-sunscreen active materials used in sunscreen products, can improve/enhance the overall UV protection provided to a person in need of sunscreen protection and who is using the sunscreen formulation according to a manufacturer's instructions. Boosters can be considered to be “inactive ingredients” when used in sunscreen formulations that provide minimal UV protection on their own—not enough to be “an active” ingredient under current regulatory schemes—but which confer at least some antioxidant or other type of functionality that, in combination with other materials in sunscreen formulations, can impart additional SPF beyond that provided by the sunscreen active ingredient(s) present in a formulation. As discussed herein, the inventors have determined that, in one implementation, addition of gadusol (and/or gadusporine) into sunscreen formulations comprising approved sunscreen active ingredients provides a substantial SPF enhancement or “boosting.”

In significant implementations, the present disclosure provides sunscreen compositions comprising one or more physical/inorganic sunscreen active ingredients in combination with gadusol in an amount suitable to convey in vivo or in vitro SPF boosting activity in the sunscreen composition. Such “boosting activity” provides an enhanced amount of SPF in a sunscreen formulation over that generated by the physical/inorganic sunscreen active on its own. While the disclosure herein discloses a number of sunscreen compositions that include ZnO, it is to be understood that the TiO₂, as a second or alternative physical/inorganic sunscreen active material that is GRASE for use in sunscreens, can be used substantially in the same manner as ZnO.

As shown by the Examples below, addition of gadusol in a “generic” cosmetic emulsion (e.g., made with ingredients in Table A and designated as “COS 1”) conveys additional SPF over “generic” compositions comprising gadusol alone e.g., made with ingredients in Table B and designated as “COS 2). Surprisingly, the SPF conferred by sunscreen formulations comprising 10% ZnO (w/w) and 0.5% (w/w) or more gadusol generates enhanced sunscreen activity over that amount that would be considered to be additive. In this regard, and as shown in Example 1 hereinafter, the amount of in vivo SPF provided by addition of 0.5, 0.75, and 1.0% gadusol in the formulations on a w/w basis are about 1.35, 1.51 and 2.0 times the SPF conferred by 10% ZnO alone.

The amount of gadusol incorporated to provide the intended in vivo sunscreen SPF boosting activity can be from about 0.25, 0.50, 0.75, 1.0, 1.25, 1.50, 1.75, or 2.0% (w/w) in the sunscreen formulation that includes a physical/inorganic sunscreen active material, where any value can form an upper or lower endpoint, as appropriate. Still further, the amount of gadusol in the sunscreen formulation can be from about 0.25 to 2.0% (w/w), or from about 0.5 to 1.0% (w/w). In these, and possibly other amounts, gadusol suitably can provide a safe and effective sunscreen boosting effect when combined with sunscreen active ingredients. In this regard, the inventors subjected gadusol to multiple safety tests appropriate prior to the introduction of a new cosmetic ingredient, and the results showed a very favorable safety profile. To summarize these safety tests, the cellular toxicity of gadusol was compared to that of a common sunscreen ingredient, oxybenzone. Vero E6 fibroblasts were used to evaluate toxicity. Gadusol was found to be less toxic than oxybenzone. Gadusol was also found not to be mutagenic in the Ames Test, nor was it toxic in the comet assay. Toxicity towards E. coli was also not found.

Moreover, since gadusol is naturally occurring in many marine and terrestrial animal and plant species, it can be hypothesized that incorporation of this material into sunscreen formulations as a booster, or for any other reason, would likely not cause negative environmental effects. At a minimum, the introduction of gadusol into marine environments resulting in potential ingestion by marine life would not be expected to be recognized as a foreign substance due to the fact that gadusol is naturally occurring in those marine animals [about 4 g gadusol per kg dry wt cod roe (Plack et al., 1981, Biochem J. 99:741-747)] and could be ingested by other organisms present in their water environments.

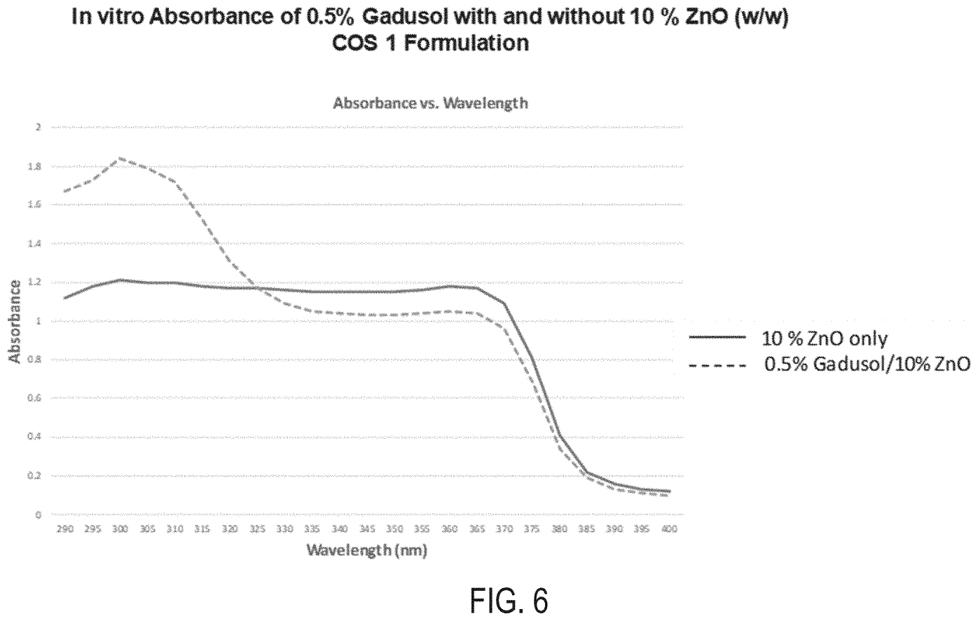

A booster activity obtained from addition of about 0.5 to about 1.0% (w/w) gadusol in a ZnO-containing sunscreen formulation, as shown in Examples 1 and 2 hereinafter, may be even more significant when considered in relation to the amount of ZnO that would need to be included in a sunscreen formulation to confer an in vivo SPF of 30 or greater. As shown in Example 1, 10% ZnO confers an in vivo SPF of about 17 with no additional active sunscreen ingredient or booster material. Furthermore, as shown in FIG. 6, the in vitro absorbance of ZnO at 10% is fairly constant from across the wavelengths primarily relevant for SPF determination of sunscreen formulations, namely 290 to 400 nm. As shown in FIG. 6, addition of 0.5% gadusol to a 10% ZnO sunscreen formulation provides significantly more absorbance from about 290 to about 325 nm, thus indicating that gadusol in combination with 10% ZnO provides a sunscreen formulation that could meet or exceed the performance criteria required for a “broad spectrum” sunscreen formulation.

In implementations, sunscreen compositions according to the present disclosure can comprise ZnO as a physical/inorganic sunscreen active ingredient and gadusol as a second ingredient. Such combination of ingredients can comprise a determined in vivo or in vitro SPF of at least about 15, 20, 25, 30, 35, 40, 45, or 50, where an intended in vivo or in vitro SPF is generated in accordance with the methodologies set out in the Examples herein. Such formulations can comprise additional ingredients that enhance or otherwise “boost” a determined in vivo SPF. Examples of such materials are discussed hereinafter.

The amount of ZnO in the sunscreen formulations comprising an amount of gadusol as a second ingredient can be less than about 25%, or less than about 20%, or less than about 15%, or less than about 10%, or about 5%, where each percentage is measured on a w/w basis in the formulation. In other implementations, the amount of ZnO can be from about 5 to about 25% on a w/w basis. Still further, the amount of ZnO can be about 2,5, 5.0, 7.5, 10.0, 12.5, 15.0, 17.5, 20.0. 22.5, or 25.0% (w/w) of the sunscreen formulation, where any value can form an upper and lower endpoint, as appropriate. Compositions comprising ZnO at any of these amounts along with an amount of gadusol as set out herein can be expected to generate an in vivo and/or in vitro SPF that is greater than the SPF of a sunscreen formulation that includes ZnO as a sunscreen active ingredient by itself (assuming negligible SPF contribution from the ingredients in the formulation).

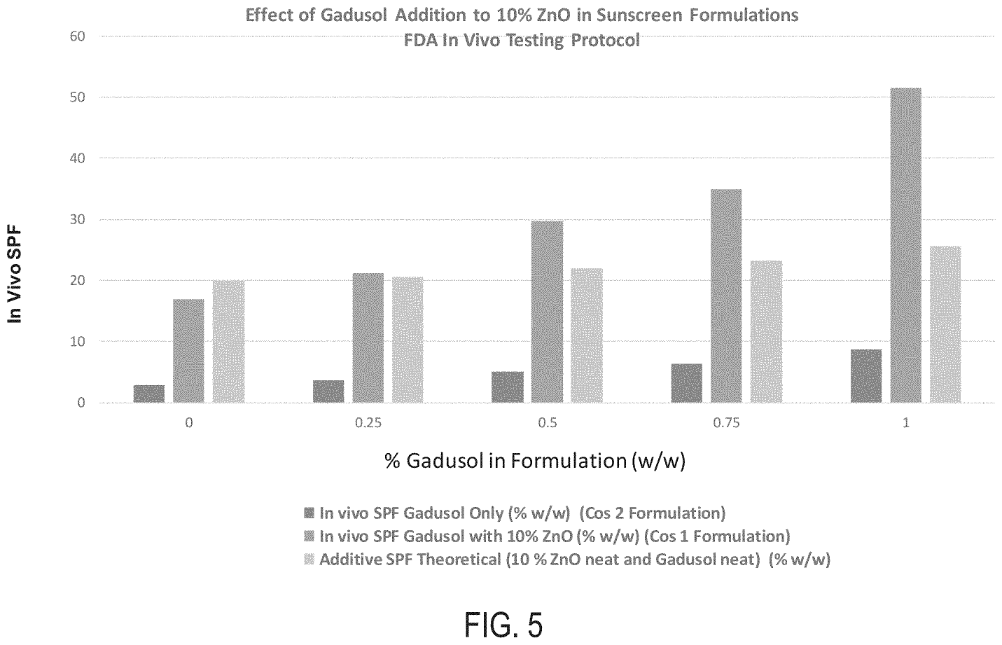

As an example, a first sunscreen composition having an amount of ZnO in any of the listed amounts can have a first determined in vivo SPF that is substantially provided by the sunscreen activity of the ZnO (assuming negligible SPF provided from the cosmetic base/formulation ingredients). A second sunscreen composition having the same amount of ZnO as the first sunscreen composition plus an amount of gadusol at least about 0.5% of gadusol can have a second determined in vivo SPF that is understood to be due to the contribution of each of the ZnO and the gadusol (assuming negligible SPF provided from the cosmetic base/formulation). While the gadusol itself provides limited in vivo SPF alone in the formulation (see Example 1), the SPF of the second sunscreen formulation can have an in vivo SPF that is greater than a sum of an in vivo SPF for the gadusol alone and the first determined SPF for the first sunscreen composition (see Table 1A and FIG. 5). In other words, the added in vivo SPF to a 10% ZnO-containing sunscreen formulation by incorporation of at least 0.5% added gadusol provides more than an additive in vivo SPF effect.

Yet further, the sunscreen compositions including ZnO and gadusol as disclosed herein appear semi-sheer or transparent when applied to a user's skin. Such an appearance would generally be unexpected for sunscreen formulations comprising ZnO having average particle sizes of 100 nm or greater, especially when applied at levels of about 10% or greater. Thus, the compositions herein not only provide excellent in vivo SPF characteristics, they also impart cosmetically acceptable aesthetics in use. The sunscreen compositions providing such intended in vivo or in vitro SPF can include ZnO at a level that will result in minimal residual whitening effect on the skin of a user upon application of an amount suitable to provide an intended SPF to a person in need thereof. The term “minimal residual whitening effect” is used herein to mean that, the formulations when evenly spread onto a skin surface at a concentration of between 1-3 mg/cm², more suitably at a concentration of 2 mg/cm², for example as set forth in the in the SPF Test Parameters, Federal Register, vol. 76, no. 117, pages 35644-35645 (Jul. 17, 2011) (previously incorporated by reference), are substantially not visible on the skin surface to the naked eye, i.e., they do not produce a whitening effect that is visible to the naked eye on one or more skin types defined by the Fitzpatrick Phototype scale discussed herein. For example, the sunscreen compositions herein cause minimal residual whitening when applied to the user's skin having a Fitzgerald Phototype of 1, 2, 3, 4, 5, or 6 when applied to the user's skin at from about 1-3 mg/cm², where each of the Phototypes can be a separate test result.

The ZnO used in the sunscreen formulations herein can substantially have an average particle size of greater than about 100 nm. The ZnO in the exemplary sunscreen formulations can comprise “conventionally sized” particles, namely as supplied in Solaveil® CZ-200 LQ (Croda). In other implementations, the ZnO can have an average particle size of less than 100 nm, for example from 50 to less than about 100 nm, or from about 25 to about 75 nm. An exemplary nano-sized ZnO material is the “Zano” line of materials from Evercare, for example, Zano® 20, which is understood to be an uncoated ZnO powder having a well-defined particle size, as is required for use thereof according to EU regulations associated with the use of nanoparticles in sunscreens. As would be appreciated, such smaller particle sizes could be beneficial for use by darker skin toned users, such as those having skin tone on the Fitzgerald Phototype Scale of 4 or greater and when the subject skin care formulation is not marketed as a “natural” sunscreen formulation. Such smaller particle size ZnO is commonly referred to as “nanoparticle” ZnO. In other implementations, the sunscreen formulations can have a mixture of “non-nano” and “nano” ZnO, that is, some ZnO that has an average particle size of greater than 100 nm and some ZnO that has an average particle size of less than 100 nm. Such particle size mixtures can be varied according to the needs of users, for example for users with lighter or darker skin tones.

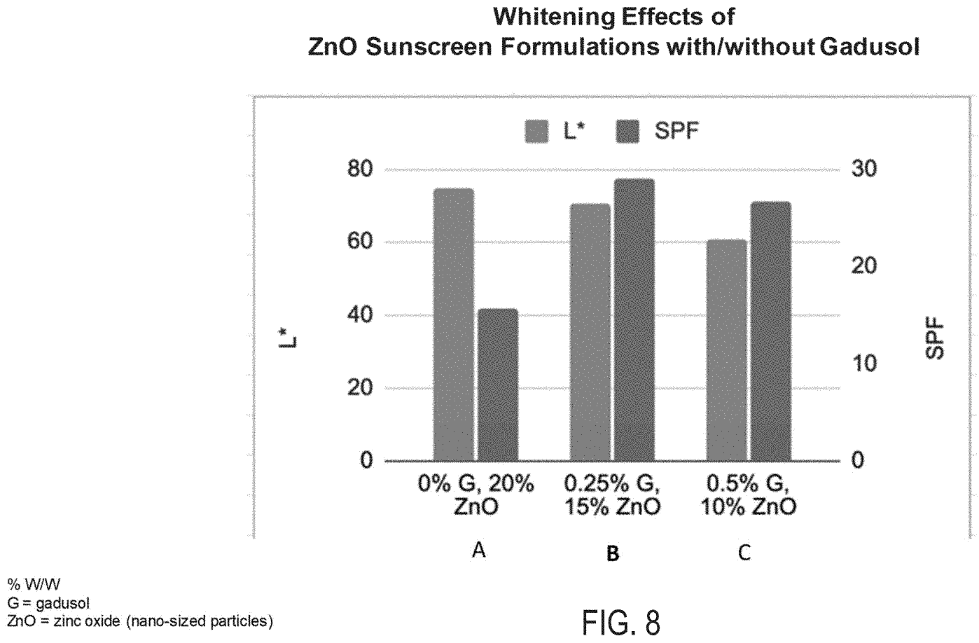

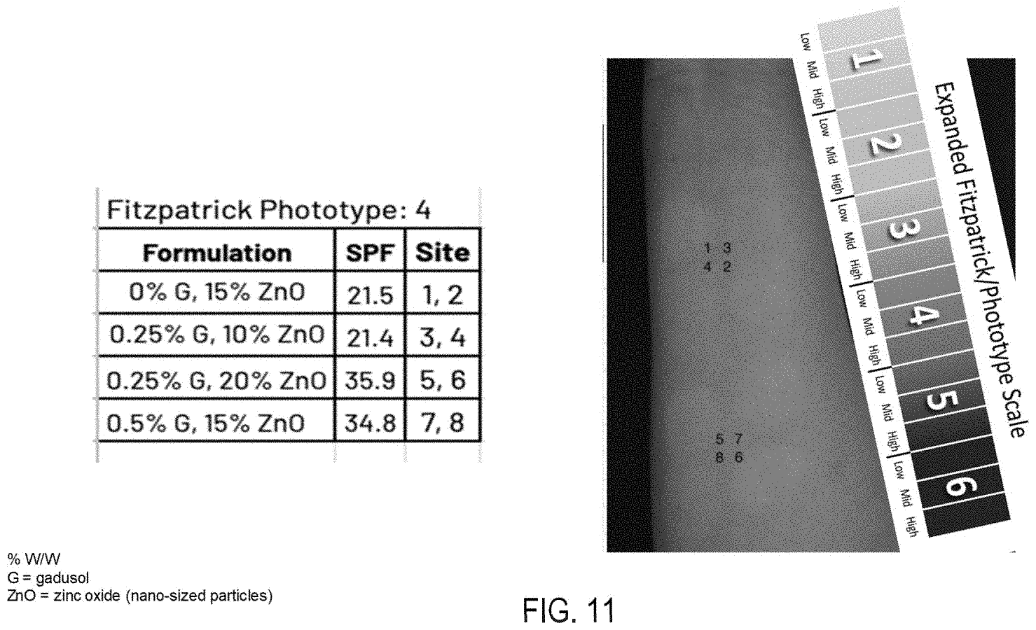

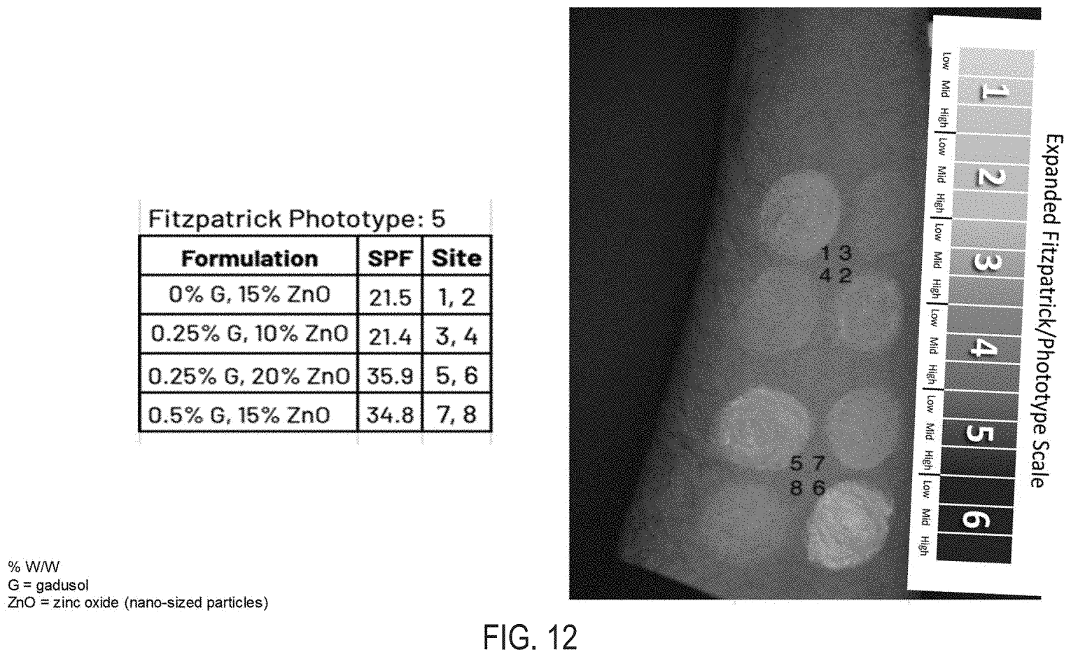

The skin whitening (or lack thereof) for various formulations according to the disclosure herein are shown in Examples 3 and 4 and the accompanying FIGS. 8-12. Both in vitro and in vivo skin whitening results are provided, with the in vivo results for whitening being shown on different Fitzgerald Scale Phototypes.

Yet further, gadusporine can be incorporated at from about 0.25, 0.50, 0.75, 1.0, 1.25, 1.50, 1.75, or 2.0% (w/w) in a sunscreen formulation that includes a physical/inorganic sunscreen active ingredient, where any value can form an upper or lower endpoint, as appropriate. Still further, the amount of gadusporine in the sunscreen formulation can be from about 0.25 to 2.0% (w/w), or from about 0.5 to 1.0% (w/w). The gadusporines A 200, B 202, and C 204 (shown in FIGS. 2A, 2B, and 2C, respectively) absorb UV radiation at different wavelengths, allowing them to be used in combination to form broad spectrum sunscreens. An example of UV spectrum coverage 400 for gadusol and gadusporine A is illustrated in FIG. 4.

Still further, the amount of physical/inorganic sunscreen active ingredient in a sunscreen formulation that, in combination with gadusporine, generates an intended in vivo or in vitro SPF of about can be from about 2.5, 5.0, 7.5, 10.0, 12.5, 15.0, 17,5, 20.0, 22.5 or 25.0% (w/w), where any value can form an upper or lower endpoint, as appropriate. The physical/inorganic sunscreen active ingredient used in such formulations can substantially have an average particle size of greater than about 100 nm or less than about 100 nm.

The disclosure further includes sunscreen compositions including a combination of gadusol and gadusporine(s). As shown in FIGS. 3 and 4, the absorbance behavior of gadusporine (shown by curve 304 of FIG. 3) is different from gadusol (shown by curve 302 of FIG. 3), meaning that their specific characteristics may be leveraged for use in various sunscreen formulations. Such combinations gadusol/gadusporine as sunscreen formulation ingredients—whether in “boosting amounts” or otherwise—can be hypothesized to provide different activity than that provided by use of either of these materials on their own. Thus, it can be expected that broad spectrum sunscreen formulations can be generated by incorporation of gadusol and one or more gadusporines A, B and/or C in sunscreen and other formulation types.

Such combinations of gadusol and gadusporine(s) can be included in sunscreen formulations with other sunscreen active ingredients, for example, physical/inorganic active ingredients as discussed herein. The amounts of gadusol and gadusporine(s) used in combination can be varied to generate a desired SPF in a range of interest. The combination of gadusol and gadusporine may be together in an amount of from about 0.25, 0.50, 0.75, 1.0, 1.25, 1.50, 1.75, or 2.0% (w/w) in a sunscreen formulation that includes a physical/inorganic sunscreen active ingredient, where any value can form an upper or lower endpoint, as appropriate. Still further, the amount of gadusol and gadusporine together in a formulation can be from about 0.25 to 2.0% (w/w), or from about 0.5 to 1.0% (w/w). Still further each amount of gadusol and gadusporine in a formulation can comprise 0.25, 0.50, 0.75, 1.0, 1.25, 1.50, 1.75, or 2.0% (w/w) in a sunscreen formulation that includes a physical/inorganic sunscreen active ingredient, where any value can form an upper or lower endpoint, as appropriate. Still further, the amount of gadusol and/or gadusporine together in a formulation can be from about 0.25 to 2.0% (w/w), or from about 0.5 to 1.0% (w/w). When used with a combination of gadusol and/or gadusporine in any of the referenced amounts, an amount of physical/inorganic sunscreen active ingredient in a sunscreen formulation that substantially causes minimal residual whitening on the skin of a user can be about 2.5, 5.0, 7.5, 10.0, 12.5, 15.0, 17,5, 20.0, 22.5 or 25.0% (w/w), where any value can form an upper or lower endpoint, as appropriate. The physical/inorganic sunscreen active ingredient used in such formulations can substantially have an average particle size of greater than or less than about 100 nm.

Yet further, the sunscreen boosting effect of gadusol and/or gadusporine can be expected to be beneficial with the use of other sunscreen active ingredients. As noted, as of late 2019, there is an open question of whether various sunscreen chemical/organic active ingredients are GRASE when used as indicated. Testing is currently underway for the following chemical/organic sunscreen actives: cinoxate, dioxybenzone, ensulizole, homosalate, meradimate, octinoxate, octisalate, octocrylene, padimate O, sulisobenzone, oxybenzone, and avobenzone. Once testing is completed on these materials, it is possible/likely that one or more of these materials will be determined to be GRASE for use in sunscreens by the FDA. The addition of gadusol and/or gadusporine to a sunscreen composition having a GRASE chemical/organic sunscreen active ingredient where the gadusol (and/or gadusporine) is added in an amount of from about 0.1, 0.25, 0.5, 0.75, 1.0, 1.25, 1.5, 1.75 or 2.0% (w/w) can be expected to provide a beneficial effect in the determined SPF of such formulation.

In further implementations, a physical/inorganic sunscreen active ingredient can also be added in an amount of from about 2.5, 5.0, 7.5, 10.0, 12.5, 15.0, 17,5, 20.0, 22.5 or 25.0% (w/w) in a GRASE chemical/organic sunscreen active sunscreen active-containing composition to further enhance the SPF of that formulation. Beneficially, the physical/inorganic sunscreen active ingredient included in these chemical/organic sunscreen active gadusol (and/or gadusporine) combination sunscreen formulations can be a non-nano (i.e., average particle size>100 nm) to enhance the non-whitening characteristics of the formulation. However, other particle size materials may be indicated in some use cases. The use of gadusol (and/or gadusporine) as an SPF booster material can allow a lower amount of either or both of chemical/organic sunscreen active and physical/inorganic sunscreen active ingredients. This can provide highly efficacious formulations, while also reducing the amount of other sunscreen active ingredients that may be indicated to have a lower safety profile at higher use amounts.

In addition to the specifically listed chemical/organic sunscreen active ingredients that are currently undergoing testing for regulatory approval in the US, it is to be appreciated that gadusol (and/or gadusporine) can be beneficial as a sunscreen booster or enhancing ingredient along with other materials that are not yet, or may never be, approved for use in the US. Since many countries do not regulate sunscreens as drugs as in the (and some other countries), ingredients intended to provide sunscreen activity may be more readily available for use by consumers. In the EU, commonly chemical/organic sunscreen active ingredients include Bemotrizinol (Tinosorb S), Bisoctrizole (Tinosorb M), Tris-Biphenyl Triazine (Tinosorb A2B), Octyl methoxycinnamate (Tinosorb OMC), Ecamsule (Mexoryl SX), among others. This disclosure is to be understood to contemplate the addition of gadusol (and/or gadusporine) as a sunscreen ingredient in an amount of from 0.1, 0.25, 0.5, 0.75, 1.0, 1.25, 1.5, 1.75 or 2.0% (w/w) to a chemical/organic sunscreen active-containing sunscreen composition, with or without a physical/inorganic sunscreen active ingredient added in an amount of from about 1.0, 2.5, 5.0, 7.5, 10.0, 12.5, 15.0, 17,5, 20.0, 22.5 or 25.0% (w/w) in the composition to further enhance the SPF of that formulation. In such broad implementations, the use of gadusol (and/or gadusporine) as a sunscreen booster can enhance the options available for the formulation of sunscreen products. Example 7 shows in vitro SPF results for 0.5% gadusol with Tinosorb as a sunscreen active ingredient along with 10% ZnO.

Yet further, various other sunscreen enhancers/boosters or other suitable materials having UVA/UVB absorber properties in the relevant wavelengths for sunscreens can be added to a gadusol and/or gadusporine-containing sunscreen formulation, with or without inclusion of physical/inorganic active ingredient. For example, polysilicone-15 can be used as a UV-absorber in sunscreens with an absorbance in the UVB range (290-320 nm) with a peak absorbance at 312 nm. Ferulic acid, ethyl ferulate, and reservatrol—which which are “natural” products in the parlance of cosmetic ingredients—have been shown to have promise for use with octinoxate as either boosters or as broad spectrum ingredients. As such, it would be understood that these, as well as other “natural” ingredients that have UVB absorbance and/or UVA absorbance characteristics could have utility in formulation of broad-spectrum sunscreen products.

Sunscreen boosters can generally be categorized as polymers, photostabilizers, or “naturals.”

Polymer sunscreen enhancers are ingredients that form a film on the surface of the skin to evenly disperse SPF actives, increase optical path length, and create a thicker film, which act to increase UV absorption of material spread on a user's skin. Use of polymer sunscreen boosters can allow use of less UV actives to deliver the same level of SPF. Examples of polymer sunscreen boosters include VP Eicosene Copolymer, Tricontanyl/PVP, and B is—Hydroxyethoxypropyl Dimethicone.

Photostabilizers are believed to act as sunscreen enhancers to quench the excited states of the UV filter and return the molecule to the ground state before it can undergo photochemical reactions that convert it to a form that does not absorb UV. Examples of photostabilizers as sunscreen boosters include Ethylhexyl Methoxycrylene, Butyloctyl Salicylate, Diethylhexyl 2,6-Naphthalate, and Trimethoxybenzylidene Pentanedione.

“Natural” SPF-booster ingredients are derived from a variety of sources and exhibit a broad spectrum of efficacy and mechanisms of action. Most naturals have their own SPF as standalone ingredients. “Natural” SPF boosters include, in non-limiting examples:

LaraCare® A200 biopolymer including Galactoarabinan;

Uvaxine® is a biotechnology active ingredient obtained by means of enzymatic glycosylation of a natural plant stilbene (piceid), using a proprietary green chemistry process;

SUN' ALGID: A combination of natural bioactive ingredients, sustainably sourced: w Pongamia glabra seed oil (Karanga seed oil, deodorized grade) provides a primary shield against UV radiation, acting as natural sunscreen thanks to its absorption ability especially for UVB but also for UVA;

Melscreen® Buriti FG: Oil extracted from the pulp of the buriti fruit (Mauritia fl exuosa) is rich in carotenoids and tocopherols, potent antioxidants capable of neutralizing free radicals and protecting the skin against damages caused by ultraviolet radiation. presents high oleic acid content (omega 9), an essential fatty acid highly nourishing and emollient for skin;

SunBoost ATB: Argania Spinosa Kernel Oil (And) Tocopheryl Acetate (And) Bisabolol Oily Material;

SunBoost ATB Natural+++: Argania Spinosa Kernel Oil (And) Tocopheryl Acetate (And) Bisabolo;

HELIONORI®: A water and concentrated active ingredient prepared from the red seaweed Porphyra umbilicalis (L.) Kützing known as “nori” in Japan. Three different MAAs are extracted selectively from Porphyra umbilicalis: palythine, porphyra 334 and shinorine; and

ASTAPLANCTON® HA: A composition including the astaxanthin molecule, which is similar to that of the ß-carotene but the small structural differences confer large differences in the chemical and biological properties of the two molecules.

As an example of such “natural” booster/enhancer materials, MAAs that have been shown to provide relevant UV absorption characteristics for use in sunscreen products include, for example, porphyra-334, shinorine and palythine, as in HELIONORI®. These materials exhibit effective multifunctional photoprotective properties in vitro and have the potential to be developed as “natural” and/or biocompatible alternatives to currently approved chemical/organic sunscreen active filters. One example of an existing product that includes liposomal porphyra-334 and shinorine is Helioguard 365®. In this regard, the SPF enhancing effects of gadusol (and/or gadusporine) can be beneficial for use in sunscreen compositions currently available or that may later be developed that incorporate various amounts of MAAs, either as sunscreen active ingredients or as boosters included with other sunscreen actives. The addition of gadusol and/or gadusporine to MAA-containing compositions as sunscreen boosters or sunscreen active adjuvants are further contemplated herein. A sunscreen composition comprising gadusol and/or gadusporine, one or more MAAs such as porphyra-334, shinorine, and/or palythine are contemplated as useful aspects of the present disclosure. Various amounts of MAAs can be included in the compositions herein to provide a desired effect, for example, in about 0.1, 0.5, 0.75, 1.0, 1.25, 1.5, 1.75, or 2.0% (w/w), with any value forming an upper or lower endpoint, as appropriate. Additionally, combinations of gadusol and/or gadusporine and MAA' s can be incorporated with “non-nano” physical/inorganic materials to provide a “natural” sunscreen formulation having an intended in vivo or in vitro SPF.

It would be appreciated that in order to effectively work as a sunscreen booster/enhancer, as well as a sunscreen active ingredient, a material also needs to be stable under the conditions associated with manufacture, storage, and use conditions of a sunscreen formulation. The term “stable” is defined as a characteristic wherein a composition retains potency for the duration of a predetermined expiration period, as defined by generally accepted pharmaceutical protocols, such as “GMP”, or “good manufacturing practices” as promulgated by various trade conventions, such as for example, the United States Pharmaceutical (USP) convention.

In this regard, the present disclosure also provides systems, methods, and compositions for providing stable formulations of gadusol and/or gadusporine containing sunscreen products. By “stable” it is meant that the potency of gadusol and/or gadusporine, as well as the stability of any other ingredients that contribute to SPF to provide the intended level of in vivo SPF in use, is retained for a period of time to generate the intended SPF for a user at a time of use. In separate implementations, the gadusol and/or gadusporine-containing sunscreen compositions herein retain at least 50%, 60%, 70%, 80% or 90% of the gadusol and/or gadusporine in active form for at least 30, 45, 60, or 90 days after formulation when stored at room temperature. Still further, the gadusol and/or gadusporine-containing sunscreen compositions herein retain an in vivo or in vitro SPF of at least 30, 40, or 50 for at least 30, 45, 60, or 90 days after formulation when stored at room temperature, where the SPF is determined as set out elsewhere herein.

In implementations, an additional amount of gadusol and/or gadusporine can be included in a formulation to account for loss of SPF activity due to deterioration prior to use. For example, if a 0.5% gadusol activity (w/w) is intended as an in-use level, and it is known or expected that 50% of the gadusol activity may be lost during storage thereof in a typical use case, an additional amount of gadusol (w/w %) can be incorporated in the formulation so that the sunscreen activity is as intended at time of use. If the formulation has a higher in vivo or in vitro SPF at use due to a lower amount of deterioration of the gadusol than expected during storage and prior to use, the sunscreen formulation can still provide the intended SPF, as well as an additional level that results from an excess amount of intact gadusol. For example, if the sunscreen formulation is intended to have an in vivo SPF of 30 at use, but it actually provides an SPF of 50 because of a presence of an additional amount of gadusol that did not deteriorate in storage, this would be expected to be a “feature” not a “bug” in a sunscreen formulation. While additional cost may be experienced from such increased gadusol active material in a formulation, the non-toxic and “natural” characteristics of additional gadusol (and/or gadusporine) could be expected to not cause any adverse effects to a user.

Still further, the stability of gadusol and/or gadusporines as needed for a sunscreen formulation may be enhanced by use of an anhydrous sunscreen formulation. Such anhydrous sunscreen formulations are disclosed, for example, in Patent Publication No. US2020/0306162, US20190183754A1, US20180015022A1, and US20150202145A1, the disclosures of which are incorporated herein in their entireties by this reference.

Yet further, the inventors have determined that the stability of aqueous gadusol can be improved when stored under acidic conditions. For example, 90% gadusol activity can be retained after 90 days at pH 2.5, where the concentration of gadusol is determined spectrophotometrically at 296 nm where it has an extinction coefficient of 21,800 M⁻¹ cm⁻¹. A standardized concentration curve can be used to determine an amount of gadusol in a volume sampled from a test solution at various time periods. Therefore, storage under acidic conditions may be useful for stabilizing gadusol generally, e.g., prior to introduction into a topical formulation. Combining gadusol with another antioxidant may be useful for extending the shelf life or stability of gadusol in a topical formulation.

Still further, antioxidants can be incorporated to improve the stability of gadusol and/or gadusporines. Addition of an antioxidant could also be helpful in increasing the stability of a gadusol and/or gadusporine-containing sunscreen formulation. For example, antioxidants, singlet oxygen scavengers, or inhibitors of singlet oxygen formation compatible with skincare products may be added to gadusol or any of the formulations described herein, such as: ferulic acid, ethyl ferulate, micah, vitamin A, E, ascorbyl palmitate, MAA, quercetin, crude natural sources or extracts of antioxidants such as seed oils (e.g., sunflower, soybean, other vegetable oils), turmeric, rosemary, ginger, saffron, and fruits or components thereof (e.g., grapes, blackberries, raspberries, strawberries).

In a further implementation, the stability of gadusol and/or gadusporine-containing sunscreen compositions could be expected to be improved with use of specialized packaging systems, such as an airless pump. Airless packaging can help increase the shelf life of the sunscreen compositions herein by substantially preventing contact with air during storage of the compositions prior to use. An airless pump dispensing container comprises a non-pressurized vacuum dispensing system that utilizes a mechanical pump. As a user depresses the pump, a disc in the bottle rises to push the product out of an exit location, typically a spout or other type of dispenser suitable for a cosmetic formulation. Once the bottle is filled, the material stored inside the bottle is typically preserved and maintained until used. Airless pumps can also provide a precise amount of sunscreen with each pump. Storage and formulation of gadusol in nitrogen-purged solutions in containers with minimal headspace can also be expected to increase gadusol and/or gadusporine stability.

The “critical wavelength” is also relevant to sunscreen formulations. The critical wavelength is the wavelength at which the sunscreen allows 10% of the rays to penetrate to reach a person's skin. The “Critical Wavelength” (λc) is thus the wavelength below which 90% of the area under the absorbance curve resides. A sunscreen with a critical wavelength over 370 nm is considered by the FDA to provide excellent UVA protection. The UV absorbance of a sunscreen product can be determined in vitro over the entire UV spectrum (290-400 nm) using substrate spectrophotometry.

To determine the critical wavelength of a sunscreen formulation, a uniform amount and thickness of sunscreen is applied to a glass slide and exposed to UV light; the absorbance of that UV radiation is then measured. The shape of a resultant absorbance curve represents the efficiency at which a sunscreen product blocks a given UV wavelength with respect to another. The “amplitude” (vertical height) of an absorbance curve reflects the degree of protection. The higher the amplitude of the curve, the greater the absorbance, and the more protection provided at that wavelength. Within the UVB portion of the spectrum (290320 nm) this amplitude correlates with the SPF. The greater the “breadth” of the curve, the more protection provided against longer wave UV radiation. In other words, the greater the “breadth” of the curve, the broader the spectrum of sun protection provided.

Sunscreen products that provide broad spectrum protection have broad (wide) absorbance curves that extend over the majority of the UV spectrum. Mathematical integration of the measured spectral absorbance from 290 to 400 nm is performed to calculate the area beneath the curve. The critical wavelength determination does not promote the false idea that UVB and UVA are separate entities, but rather are part of the continuous electromagnetic spectrum. The critical wavelength for a UVB sunscreen is less than that for a sunscreen that protects against both UVB and UVA. A higher critical wavelength ensures more UV protection, especially protection from longer wavelength UVA rays. For example, a sunscreen with a critical wavelength of 370 nm provides more protection against UVA rays than a sunscreen with a critical wavelength of 350 nm. Calculation of the critical wavelength provides a convenient and reproducible method of evaluating the breadth of UV protection. When combined with SPF testing, the critical wavelength provides a simple and explicit means of communicating broad-spectrum photoprotection to the consumer.

A critical wavelength of >370 nm is a rigorous minimum that sunscreen products should achieve to be labeled as “broad-spectrum.” The combination of SPF rating and critical wavelength provides a complete description of a product's inherent photoprotective characteristics; SPF describes the amplitude of protection (at a given application thickness), and critical wavelength provides a reliable measurement of a product's absorption capability over the entire UV spectrum. Accordingly, in some implementations, the sunscreen formulations including gadusol and/or gadusporine and physical/inorganic sunscreen active ingredients can be considered to be “broad spectrum” products, that is, they comprise a critical wavelength of at least 370 nm. Yet further, the ratio of UVA/UVB protection provided is at least 0.3 or greater.

The disclosure herein also addresses selecting a target amount of UVA and UVB protection, such as an SPF or a critical wavelength, to be provided or generated from a sunscreen formulation when it is worn as indicated on a person in need of sun protection. A sunscreen formulation can be generated comprising a plurality of ingredients, where the plurality of ingredients comprises gadusol and/or gadusporine in an amount of at least about 0.25% on a w/w basis in addition to an amount of physical/inorganic sunscreen active ingredient.

Such sunscreen formulations comprising intended SPF protection values, in one implementation, does not incorporate any of the materials designated as “chemical/organic sunscreen actives,” as such term is known to those of ordinary skill in the art. In a non-limiting example, a sunscreen formulation comprising a physical/inorganic sunscreen active ingredient— that is, either or both of ZnO or TiO₂—can be combined with either or both of gadusol or gadusporine in an amount sufficient to generate a SPF value of interest. One or more additional ingredients can be selected to generate increased SPF, such as would be provided by addition of a sunscreen enhancer/booster ingredient as discussed hereinabove. When the collection of ingredients that collectively generates the intended SPF or critical wavelength value for the formulation does not include any chemical/organic sunscreen active sunscreen ingredients and the physical/inorganic sunscreen ingredient is “non-nano,” such formulation might be designated to be a “natural” sunscreen formulation in the context of a manufacturer's product line.

Gadusol and/or gadusporine can beneficially be dispensed in the water phase of an o/w or w/o emulsion. The inventors have determined that by incorporating this material in the water phase, loss of activity of gadusol and/or gadusporine prior to use might be mitigated. Antioxidant materials that may be included—whether as stability-enhancers for the gadusol and/or gadusporine or as skin-active ingredients—can also be included in some implementations.

When a physical/inorganic sunscreen active ingredient is included in a gadusol (and/or gadusporine)-containing formulation, methods of formulating emulsion-based compositions including physical/inorganic sunscreen active ingredients can be useful. O/W emulsions can provide a lighter and more cosmetically desirable feel that consumers better appreciate in a typical use case. If water resistance is desired for an O/W formula, then a water-resistant polymer can be added to the formulation. The gadusol and/or gadusporine-containing sunscreen formulations are water resistant, as such term is defined elsewhere herein.

As would be appreciated, in an O/W emulsion, emulsifiers will surround the oil with their hydrophobic moiety oriented toward the oil, thus forming a protective layer so that the oil molecules cannot coalesce. For W/O emulsions, the emulsifier will maintain the water as an internal phase in an oil continuous phase. Depending on the type of emulsion, the emulsifiers may be anionic, nonionic, or cationic. A suitable emulsifier for use in the gadusol and/or gadusporine-containing compositions of the present disclosure are those that can maintain consistent emulsion characteristics such as particle size, appearance, texture, and viscosity, substantially constant for as long a period as possible since by their very nature all emulsions due to their metastable nature will eventually separate into their constituent oil soluble and water-soluble components. Moreover, consistent with the “natural” characteristics of some of the implementations of the present disclosure, non-petroleum derived ingredients can be beneficial.

Whether a physical/inorganic sunscreen active ingredient (i.e., ZnO and/or TiO₂) is coated or uncoated should be assessed in selecting the various ingredients suitable for the formulations herein. It should also be mentioned that a physical/inorganic active ingredient-containing may not be a benign ingredient when included in an emulsion. For example, acrylate-derived polymers are sensitive to Zn2+, as it forms complexes that can result in disruption of the polymer matrix and loss of stability. This effect can be even more pronounced with the uncoated zinc oxide. For example, Zn2+ can interact with rheology modifiers, film formers and emulsifiers. Using a coated ZnO material can help minimize the ion migration into the water phase of the emulsion. Chelating agents such as disodium EDTA may also help reduce the extent of these interactions, as they help bind some of the ions that may form in the formulation.

While there are many methods to prepare emulsions, in an illustrative example, the ZnO can be added to the oil phase during preparation. The ZnO can be added in powder form or in a pre-dispersed formulation including other cosmetic ingredients. If the latter, such cosmetic ingredients can be accounted for in the formulation instruction. A coated material can minimize the ion migration and can allow the formulator more choices of non-polar emollients that can be used to create the dispersion. It is possible to use uncoated material, but extra precaution should be taken to mitigate ion migration in the formulation, since this could cause instability in an emulsion over time. Dispersing in water can be considered for untreated powders. Premixing with a humectant or water-soluble emollient can improve dispersion of powders.

Sunscreen formulations can include ingredients like petrolatum, mineral, dimethicone, and/or plant-based dimethicone substitutes, such as coco-caprylate/caprate. Humectants can also be added, such as urea, glyceryl triacetate, propylene glycol, hexylene glycol, and butylene glycol. Humectants can also be derived from natural sources, like glycerin, honey, aloe vera gel or liquid, sorbitol (derived from sugar cane), lactic acid, and hydrolyzed wheat, baobab, and rice proteins, urea, glyceryl triacetate, propylene glycol, hexylene glycol, and butylene glycol. Emollients can be added to improve the feel of a sunscreen or other product on the skin, such as by reducing tackiness and greasiness. A non-limiting list of possible emollients can include coconut oil, cetyl esters, and certain silicones.

Nonionic emulsifiers and rheology modifiers can be beneficial for use with physical/inorganic sunscreen active ingredients. Some thickeners useful are ingredients that include xanthan gum, clays and cellulose-based thickeners including hydroxyethyl cellulose and hydroxypropylmethyl cellulose. Some examples of nonionic emulsifiers for 0/W systems are stearates, glucosides and oleates. When it comes to density loading, at high loads of zinc oxide, the oil droplets can become denser than the water phase, which causes agglomeration and settling of droplets. Therefore, it can be beneficial to increase the concentration of emulsifiers and rheology modifiers in some implementations.

Exemplary instructions for making sunscreen formulations including gadusol and/or gadusporine are set out below in the Examples. As would be appreciated, the various parameters associated with formulating an acceptable composition comprising gadusol and/or gadusporine may vary according to the use case, desired organoleptic properties, interactions between ingredients, etc.

Gadusol and/or gadusporines can also be formulated with other materials that have antioxidant activity, with an emphasis generally being placed on those derived from natural sources. Natural antioxidants used in the cosmetic industry include various substances and extracts derived from a wide range of plants, grains, and fruits, and are capable of reducing oxidative stress on the skin or protecting products from oxidative degradation. The use of plant antioxidants is increasing and may eventually replace the use of synthetic antioxidants. A natural antioxidant can be a single pure compound/isolate, a combination of compounds, or plant extracts; these antioxidants are widely used in cosmetic products. Vitamins (e.g., B, C, E, and K) can also comprise antioxidant activity. Polyphenols, such as those included in reservatrol/grapeseeds, ferulic acid/broccoli, cucumins/turmeric, and aloe vera also can be added for an antioxidant effect. In some implementations, such antioxidants may have utility in enhancing the stability of the gadusol and/or gadusporine in a formulation.

In particular implementations, the formulations herein comprising either or both of gadusol and gadusporine can substantially exclude any ingredients that are derived from petroleum or petroleum-containing products. Yet further, substantially all of the ingredients in the formulations herein can be derived from biological sources with the exception of any materials that are physical/inorganic sunscreen active ingredients and any other inorganic ingredients (e.g., silica, salt, etc.).

In non-limiting examples, “natural” ingredients that can be included in the formulations including gadusol and/or gadusporine can include: Candelilla/Jojoba/Rice Bran, Polyglyceryl-3 Esters, Glyceryl Stearate, Glycerin, Cetearyl Alcohol, Sodium Stearoyl Lactylate, Caprylic/Capric Triglycerides, Butyrospermum Parkii Butter, Vitis Vinifera Seed Oil, Prunus Armeniaca Kernel Oil, Palmitic/Stearic Triglycerides, Cera Alba, Aloe vera extract, Amaranthus Caudatus Seed Oil, Hippophae Rhamnoides Extract, Argania Spinosa Oil, Helianthus Annuus Seed Oil, Lycium Barbarum Fruit Extract, Mimosa Tenuiflora Bark Extract, Sodium Benzoate, Potassium Sorbate, Mixed tocopherols, Cocos Nucifera Oil, Prunus Amygdalus Dulcis Oil, Musa Sapientum Fruit Extract, Olea Europaea Leaf Extract, Sambucus Nigra Flower Extract, Camelia Sinensis Leaf Extract, Glycyrrhiza Gia bra Extract, Cyamopsis Tetragonoloba Gum, Glycyrrhetinic Acid, Phytic Acid, Citrus Aurantifolia Peel Oil, Helianthus Annuus Seed Oil, resveratrol, stilbenes generally, flavonoids as a class, and Rosmarinus Officinalis Leaf Extract, among other ingredients that can suitably provide a gadusol and/or gadusporine formulation that provides the desired properties and activities to a user in need thereof.

As would be appreciated, gadusol and/or gadusporine can also be included in formulations that do not comprise sunscreen active ingredients, such as when used in an antioxidant topical skin formulation. In this regard, gadusol and/or gadusporine-containing formulations can have utility as “cosmeceutical” products. While the term “cosmeceutical” has no meaning under the law, and while the U.S. Food, Drug, and Cosmetic Act (FD&C Act) does not recognize the term “cosmeceutical,” the cosmetic industry uses this word to refer to cosmetic products that have medicinal or drug-like benefits. To this end, the term can be used to define a category of skincare products containing ingredients that are backed by scientific evidence that demonstrates relevance to one or more biological properties associated with a user's skin, with a goal of enhancing a user's skincare regimen and providing noticeable, positive changes to the appearance of the user's skin. Typical active ingredients in cosmeceutical products can include vitamins, antioxidants, and/or botanical extracts.

In addition to a sunscreen booster or SPF enhancing effect, gadusol and/or gadusporines also have the capacity to absorb UV radiation and may act as free radical scavengers. Therefore, gadusol and/or gadusporines may also suitably be added to cosmetic formulations as antioxidant ingredients and may have therapeutic properties, that is, they may be considered to be “cosmeceuticals.” Antioxidants neutralize free radicals, which are produced by sunlight, street air, auto pollution and other environmental factors. Antioxidant based products can be as important as sunscreen-containing products in battling aging and skin cancer. It would therefore be understood that gadusol and/or gadusporine can have utility as antioxidant ingredients in topical treatments, skin care creams, cosmetics and the like. The proposed antioxidant behavior of gadusol and/or gadusporine can provide an additional naturally derived antioxidant material when formulated to provide a suitably stable cosmetic formulation. The amounts of gadusol and/or gadusporine used in antioxidant formulations can be from about 0.1, 025, 0.5, 0.75, 1.0, 1.25, 1.5, 1.75, or 2.0% (w/w), where any value can form an upper or lower endpoint, as appropriate. The amount of gadusol and/or gadusporines used in antioxidant formulations can be from about 0.1 to 2.0% (w/w), or from about 0.5 to about 1.0% (w/w).

### EXAMPLES

The following examples are provided to illustrate sunscreen and topical skin care formulations that may be used. While the examples are provided as sunscreen formulations, it is contemplated that topicals that do not meet regulatory definition(s) of sunscreens may be produced via inclusion of gadusol, gadusporine, MAAs, or a combination thereof, i.e., with or without another approved sunscreen agent, antioxidant, etc. This may include topicals or other formulations that are not marketed as sunscreens but nonetheless would benefit from inclusion of gadusol, gadusporine, or MAAs, e.g., antioxidant creams, lotions, etc.

### Test Protocols

A. SPF Determination of Gadusol-containing Formulations with and without Physical/Inorganic Sunscreen Active

A formulation denoted COS 1 containing 10% ZnO (non-nano) and 0.5% gadusol was prepared according to the following component listing in Table A.

TABLE A
COS 1 FORMULATION (INCLUDES 10% “NON-NANO” ZnO)

| INCI Name | Phase | Trade Name | Supplier | % (w/w) |
|---|---|---|---|---|
| Water (Aqua) | A | N/A | N/A | 54.74 |
| Sodium Phytate | A | Dermofeel PA-12 | Dr. Stratemans/Kinetik | 0.05 |
| Xanthan Gum | A | Keltrol CG-SFT | CP Kelko/IMCD US | 0.15 |
| Microcrystalline Cellulose; | A | Avicel PC 611 | FMC/Earth Supplied | 1.50 |
| Cellulose Gum | | | Products | |
| Glycerin | A1 | Glycerin 99.7% USP | DeWolf/Azelis | 1.00 |
| | | Azelis | | |
| Propanediol | A1 | Zemea Propanediol | DuPont/E s sential | 3.00 |
| | | | Ingredients | |
| Ethylhexylglycerin | A1 | Sensiva SC-50 | Schulke & Mayr | 1.00 |
| Capylyl Glycol; | A1 | Sensiva SC-10 | Schulke & Mayr | 1.00 |
| Ethylhexylglycerin | | | | |
| Hydrogenated Lecithin | B | Lecinol S-10 | Barnet | 0.20 |
| Cetearyl Alcohol; Coco- | B | Montanov 82 | SEPPIC | 4.00 |
| Glucoside | | | | |
| Coco-Glucoside; Coconut | B | Montanov S | SEPPIC | 1.50 |
| Alcohol | | | | |
| Cetearyl Alcohol | B | SP Crodocol CS50 | Croda | 1.00 |
| | | MBAL-PA-(MH) | | |
| Behenyl Alcohol | B | Lanette 22 | BASF/DeWolf | 0.25 |
| Zinc Oxide; | B | Solaveil CZ-200-LQ- | Croda | 17.50 |
| Caprylic/Capric | | (WD) | | |
| Triglyceride; | | | | |
| Polyhydroxystearic Acid; | | | | |
| Isostearic Acid (of which | | | | |
| Zinc Oxide is 10% w/w) | | | | |
| Heptyl Undecylenate | B | Lexfeel Natural | Index | 1.50 |
| Diheptyl Succinate Capryol | B | Lexfeel N5 | Index | 7.00 |
| Glycerin/ Sebacic Acid | | | | |
| Copolymer | | | | |
| Diheptyl Succinate Capryol | B | Lexfeel N100 | Index | 2.00 |
| Glycerin/ Sebacic Acid | | | | |
| Copolymer | | | | |
| Sorbitan Oleate | B | Span 80-LQ-(AP) | Croda | 0.10 |
| Silica | B | MSS-500W | Kobo | |
| Silica | B | MSS-500/3H | Kobo | 0.5 |
| Gadusol | B | Gadusol Laboratories | Synthesized per | Varied |
| | | | disclosure | |
| Citric Acid | C | Citric Acid USP 20% | Spectrum | q.s. |
| | | solution | | |

Preparation Instructions:

Step 1: For Phase A materials, dissolve Dermofeel PA-12 in water. When dissolved add each additional Phase A ingredient individually. Mix phase A until uniform.

Step B: Premix Phase Al ingredients. Once uniform, add Phase Al to Phase A and heat to 75 to 80 deg. C, while stirring.

Step C: Combine Phase B materials and heat to 75-80 deg. C. Mix until uniform. o Step D: While using homogenizer, add Phase B slowly to mixture of Phase A/A1 and mix until uniform. Continue homogenizing for about 10 minutes at 4000 rpm on Silverson at 75 to 80C.

Step E: Remove from homogenizer and cool down with mixing to 40 to 45 deg. C.

Step F: Add citric acid to adjust pH 6.5-7.5.

Step G: Homogenize for 5 minutes at 4000 rpm.

Step H: Inspect after 12-18 hours. If separation occurs, conduct additional homogenization step.

Variations of the above formulation included different amounts of gadusol (0.0, 0.25, 0.5, and 0.75, and 1.0% (w/w)), as set out in Example 1.

A cosmetic formulation denoted COS 2 containing gadusol in various amounts as shown in Table B below was prepared according to the following component listing.

TABLE B
COS 2 FORMULATION (OMITS 10% “NON-NANO” ZnO)

| INCI Name | Phase | Trade Name | Supplier | % (w/w) |
|---|---|---|---|---|
| Water (Aqua) | A | N/A | N/A | 51.20 |
| Sodium Phytate | A | Dermofeel PA-12 | Dr. Stratemans/Kinetik | 3.05 |
| Xanthan Gum | A | Keltrol CG-SFT | CP Kelko/IMCD US | 3.20 |
| Microcrystalline | A | Avicel PC 611 | FMC/Earth Supplied Products | 1.50 |
| Cellulose; Cellulose | | | | |
| Gum | | | | |
| Glycerin | A | Glycerin 99.7% USP | DeWolf/Azelis | 1.00 |
| | | Azelis | | |
| Propanediol | A1 | Zemea Propanediol | DuPont/Essential Ingredients | 3.00 |
| Ethylhexylglycerin | A1 | Sensiva SC-50 | Schulke & Mayr | 1.00 |
| Capylyl Glycol; | A1 | Sensiva SC-10 | Schulke & Mayr | 1.00 |
| Ethylhexylglycerin | | | | |
| Hydrogenated | B | Lecinol S-10 | Barnet | 3.20 |
| Lecithin | | | | |
| Cetearyl Alcohol; | B | Montanov 82 | SEPPIC | LOO |
| Coco-Glucoside | | | | |
| Coco-Glucoside; | B | Montanov S | SEPPIC | 1.50 |
| Coconut Alcohol | | | | |
| Cetearyl Alcohol | B | SP Crodocol CS50 | Croda | 1.00 |
| | | MBAL-PA-(MH) | | |
| Behenyl Alcohol | B | Lanette 22 | BASF/DeWolf | 3.25 |
| Caprylic/Capric | B | Liponate GC | Vantage (formerly LIPO) | 7.50 |
| Triglyceride | | | | |
| Heptyl Undecylenate | B | Lexfeel Natural | Inolex | 1.50 |
| Diheptyl Succinate | B | Lexfeel N5 | Inolex | 7.00 |
| Capryol Glycerin/ | | | | |
| Sebacic Acid | | | | |
| Copolymer | | | | |
| Diheptyl Succinate | B | Lexfeel N100 | Inolex | 2.00 |
| Capryol Glycerin/ | | | | |
| Sebacic Acid | | | | |
| Copolymer | | | | |
| Sorbitan Oleate | B | Span 80-LQ-(AP) | Croda | 3.10 |
| Silica | B | MSS-500W | ECobo | |
| Silica | B | MSS-500/3H | ECobo | 3.5 |
| Gadusol | B | Gadusol | Synthesized per disclosure | Varied |
| | | Laboratories | | |
| Citric Acid | C | Citric Acid USP | Spectrum | q.s. |
| | | 20% solution | | |

Preparation instructions mirrored those for COS 1 above. Amounts of gadusol in the various formulations are shown in Example 2.

### B. In Vivo SPF Determination

An in vivo SPF determination was conducted according to FDA, 21 CFR Sec. 201.327, subpart (i), SPF Test Procedure, Sunscreen Drug Products for Over-the-Counter Human Use, Final Monograph, Federal Register, Vol. 76, No. 117, Jun. 17, 2011, the disclosure of which is incorporated herein in its entirety by this reference. Each in vivo test for a sunscreen formulation was tested on three different subjects.

For subject enrollment, the test subjects reported to the testing laboratory and received a complete explanation of the study procedures. Those who participated signed a written, witnessed consent form, and a permission to release personal health information form and provided a brief medical history. The technician did a final examination of the subject's back, between the belt-line and shoulder blades and determined their suitability to participate in this study.

A Xenon Arc Solar Simulator lamp, which has a continuous light spectrum in the UVA and UVB range (290-400 nanometers) was utilized for the in vivo light source. The spectral output of the solar simulator was filtered so that it meets the spectral output requirements for testing Sunscreen Drug Products for over-the-counter human use; FDA Final Monograph, 21 CFR Part 201.327 (i)(1), UV Source, Federal Register, Vol. 76, No. 117, Jun. 17, 2011 and the International Sun Protection Factor (SPF) Test Method, May 2006, the disclosure of which is incorporated herein in its entirety by this reference.

For a baseline value, a series of 5 UV radiation doses expressed as Joules/square meter, increasing in 25% increments, was administered to two unprotected separate locations on each test subject's back, just below the shoulder blades and above the belt-line, to determine the initial unprotected MED (MEDu).

The MEDu was administered in the following 5 dose series, with X representing the amount of UV energy projected to produce the test subject's MEDu (Table C).

TABLE C
MEPu Dose Administration

| | Amount of UV Energy Projected to |
|---|---|
| Application | Produced Subject’s MEDu |
| Dose 1 | 0.64x |
| Dose 2 | 0.80x |
| Dose 3 | 1.00x |
| Dose 4 | 1.25x |
| Dose 5 | 1.56x |

On Day 2, subjects returned to the testing laboratory within 16 to 24 hours following completion of the MEDu doses for evaluation of their responses, and to determine each subject's unprotected MED (MEDu). A subject's Minimal Erythemal Dose (MED) was the quantity of erythema effective energy, or dose corresponding to the first site that produced the first unambiguous erythema reaction with well-defined borders. The following grading scale used in this study for determining a MED (+) response.

− No perceptible erythemal response

? Barely perceptible erythemal response

+ Unambiguous erythema reaction with well-defined borders

++ Moderate erythema with sharp borders

+++ Dark red erythema with sharp borders

After determination of MEDu for each test subject, the sunscreen test formulations were applied. For each in vivo test sunscreen formulation application, two test areas (10 cm x 5 cm), 50 square centimeter rectangles, were drawn in the designated locations on a subject's back, (between the beltline and the shoulder blade) using a template and an indelible marker. The technician applied the test formula in one of the test areas and the FDA standard sunscreen in the adjacent test area. The sunscreens were applied by “spotting” the product across the test area and gently spreading, using a finger cot (as specified in FDA, 21 CFR 201.327, subpart (4)(iii), until a uniform film was applied to the entire test area. A product density of 2 mg/cm² was delivered to the test area. To accomplish this, the technician weighed an amount in excess of 100 mg, to allow for the residual amount left on the finger cot (approximately 10%). The test products were permitted to dry a minimum of 15 minutes prior to the Static UV exposures on the standard sunscreen and sponsor test sample.

MEDp UV Dose was administered by the technician via a series of 5 UV radiation doses expressed as Joules/square meter, as specified in FDA, 21 CFR, Sec. 201.327, subpart (5)(iii), progressively increasing in increments of 15 or 25 percent, determined by the previously established MEDu from Day 1 and the expected SPF range of the test product. The MEDp was administered in the following 5 dose series with X representing the expected amount of UV energy required to produce a MEDp.

TABLE D
MEPp Dose Administration Expected SPF 16.3
(FDA Standard Statistic)

| Light | Amount of UV Energy Based on |
|---|---|
| Application | Subject’s Determined MEDu |
| Dose 1 | 0.76x |
| Dose 2 | 0.87x |
| Dose 3 | 1.00x |
| Dose 4 | 1.15x |
| Dose 5 | 1.32x |

Also on Day 2, the technician administered a second timed series of 5 UV doses, increasing in 25% increments to an unprotected area of the subject's back to determine the subject's second day MEDu according to Table D above

For Day 3 evaluation, subjects returned 16 to 24 hours following completion of the UV doses from Day 2. The MED for all sites that received UV doses, both protected and unprotected areas was evaluated.

In vivo SPF grading details:

The study was conducted in a double-blinded manner. Neither the test subjects nor the designated staff member who evaluated the MED responses knew which sunscreen formulation was applied to which site or what doses of UV radiation were administered, as he was not the technician who applied the sunscreen test products or administered the doses of UV radiation.

The grader evaluated and recorded the MED responses on both the unprotected and protected test sites under the following conditions:

The source of illumination was a warm white fluorescent light bulb that provides a level of illumination of at least 450 lux at the test site.

The test subject was seated when evaluated, the same as when the test sites were irradiated.

In vivo SPF values were calculated for both the test product and the FDA standard using FDA, 21 CFR, 201.327,subpart (i) (6), Determination of SPF, by calculating the ratio of the MEDp value produced in the sunscreen protected sites to the MEDu produced in the unprotected test area, for each individual using the following calculation:

MEDip/MEDiu=in vivo SPF

Data from three (3) subjects was used for calculating the test product's “projected” label SPF value. The mean SPF value (x) and the Standard Deviation (s) for these subjects was computed. Based on a subject test panel, the upper 5-percent point from the student distribution table (denoted by t) with n-1 degrees of freedom was obtained. The quantity A was computed using the Formula A=is/Square root n (with n representing the number of test subjects). A label SPF value was calculated by determining the largest whole number less than X −A. Any test product with a label SPF less than 2 is not a sunscreen drug product and will not display an SPF value.

### C. In Vitro SPF Determination

SPF was also evaluated using an in vitro method that utilizes a synthetic/simulated skin as a test substrate. In this regard, VitroSkin® is an advanced testing substrate that effectively mimics the surface properties of human skin. It has been formulated to have topography, pH, critical surface tension, chemical reactivity and ionic strength that is similar to human skin. The procedure for in vitro testing was conducted according to a standard protocol. For testing, the test product was applied in a known amount to the substrate at an application density of 2 mg/cm². The in vitro SPF scores were determined using a Labsphere UV-1000 Ultraviolet Transmittance Analyzer. Spectral scans of absorbance of the artificial skin treated with a sample sunscreen formulation were performed in the UV region of 290-400 nm. The in vitro SPF was then calculated using the absorbance scan results. At least 5 in vitro SPF values were recorded for each example formulation and the averages calculated for reporting.

### Example 1: In Vivo SPF Determination of Gadusol-Containing Formulations with and without Physical/Inorganic Sunscreen Active

TABLE 1A
Sunscreen Booster Effect of Gadusol in 10% ZnO
Sunscreen Compositions

| | | | | | Expected | % Boost = |
|---|---|---|---|---|---|---|
| Formula | | ZnO | | Observed | in vivo | (obs SPF/ |
| Code | Sample | (% | Gadusol | in vivo | SPF-if | exp |
| (1) | Code | w/w) | (% w/w) | SPF | additive | SPF) × 100 |
| COS2 | GLAB16 | 0 | 0 | 2.93 | N/A | N/A |
| COS2 | GLAB17 | 0 | 0.25 | 3.6 | N/A | N/A |
| COS2 | GLAB18 | 0 | 0.5 | 5.11 | N/A | N/A |
| COS2 | GLAB19 | 0 | 0.75 | 6.3 | N/A | N/A |
| COS2 | GLAB33 | 0 | 1 | 8.74 | N/A | N/A |
| COSI | GLAB21 | 10 | 0 | 16.92 | N/A | N/A |
| COS1 | GLAB31 | 10 | 0.25 | 21.11 | 20.52 | 102.9 |
| COS1 | GLAB22 | 10 | 0.5 | 29.7 | 22.03 | 134.8 |
| COS 1 | GLAB32 | 10 | 0.75 | 35 | 23.22 | 150.7 |
| COS 1 | GLAB34 | 10 | 1 | 51.44 | 25.66 | 200.5 |

1) The compositions denoted Cos1 and Cos2 included slightly different ingredients due to the incorporation of 10% ZnO in Cos1. The ingredient listings are provided in Tables 1A and 1B. For varied amounts of gadusol in each formulation, a 5% “hole” was generated in the base solution and water q.s. was added to generate the final formulations.

Table 1A above and FIG. 5 herewith demonstrate that gadusol added at 0.5% (w/w) and greater substantially increases the in vivo SPF of an O/W sunscreen formulation incorporating 10% (w/w) non-nano ZnO, beyond an expected additive effect. Specifically, samples GLAB17, GLAB18, GLAB19, and GLAB33 show the contribution of gadusol alone to SPF at concentrations ranging from 0.25 to 1% (w/w), whereas sample GLAB21 shows the contribution of 10% (w/w) ZnO alone. The last four rows of the table show both the observed and expected in vivo SPF activity for samples containing gadusol at 0.25 to 1% (w/w) combined with 10% (w/w) ZnO. The final column in the table shows the “% boost” for these latter samples defined as:

(observed in vivo SPF activity/expected in vivo SPF activity)×100.

Gadusol added from 0.5 to 1% (w/w) to a formulation containing 10% ZnO provided an SPF boost ranging from 135 to 200%.

### Example 2: In Vitro SPF Determination of Gadusol-Containing Formulations with and without Physical/Inorganic Sunscreen Active

In another example, gadusol was tested by the in vitro method using the Vitroskin substrate in the methodology discussed above. The COS 1 formulation having 0.0 and 0.5% (w/w) gadusol and the COS2 formulations were tested with 0, 0.25, 0.5% (w/w) gadusol.

TABLE 2A
IN VITRO SPF RESULTS FOR GADUSOL-CONTAINING
FORMULATIONS WITH AND WITHOUT PHYSICAL/INORGANIC SUNSCREEN
ACTIVE

| | | | | | Expected | |
|---|---|---|---|---|---|---|
| | | ZnO | | | in vitro | % Boost = |
| | Formulation | (% | Gadusol | Observed | SPF-if | (obs SPF/exp |
| Base | Code | w/w) | (% w/w) | in vitro SPF | additive | SPF) × 100 |
| Cos2 | GLABS16 | 0 | 0 | 1.1 ± 0 | N/A | N/A |
| Cos2 | GLABS17 | 0 | 0.25 | 3.9 ± 0.4 | N/A | N/A |
| Cos2 | GLABS18 | 0 | 0.50 | 3.6 ± 0.6 | N/A | N/A |
| Cos1 | GLABS21 | 10 | 0 | 13.9 ± 1.1 | N/A | N/A |
| Cos1 | GLABS22 | 10 | 0.5 | 26.6 ± 1.8 | 15.8-19.2 | 165-222 |

As shown in the above Table 2A, gadusol by itself shows minimal in vitro SPF activity (2.5-2.8 SPF units when the activity of the formulation matrix is subtracted, 1.1), whereas 0.5% (w/w) gadusol generates an almost 50% greater SPF when added to a composition containing 10% ZnO. This further shows the effective booster effect of gadusol. To this end, from the individual in vitro SPF contributions of each 10% (w/w) ZnO and 0.5% (w/w) gadusol, one would expect a total in vitro SPF of about 16-19 for GLABS22. Instead, the observed in vitro SPF of this formulation is about 25-28 units which corresponds to a 165-222% boost. This result confirms that gadusol added to a physical/inorganic sunscreen active acts as an effective SPF booster/enhancer.

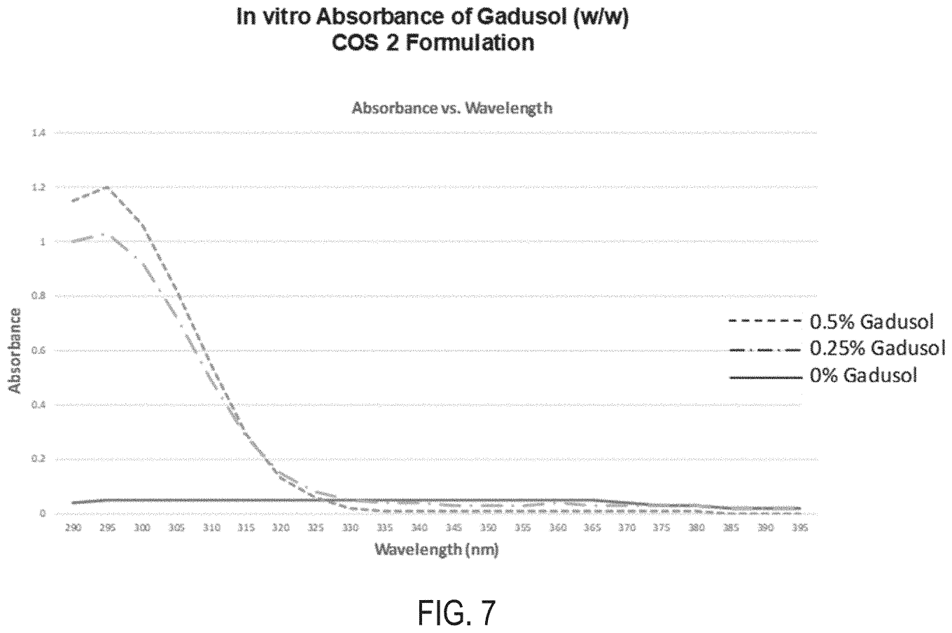

Data obtained from this in vitro testing also show that when included with 10% ZnO, gadusol provides a broad spectrum sunscreen formulation. In this regard, as shown in FIG. 6, the absorbance of the formulation designated “GLABS22” was significantly greater in the range of 290 to 320 nm than the corresponding formulation “GLAB S21” that did not show the same level of absorbance at these same wavelengths. The absorbance of gadusol is shown in FIG. 7 as being attributable to gadusol and not other ingredients in the formulation.

### Example 3: ZnO (Nanoparticle) and Gadusol Sunscreen Formulation

The following formulation was prepared with a 15% ZnO nanoparticle-containing sunscreen formulation with 0.5% gadusol (w/w).

TABLE 3A
ZnO (NANOPARTICLE) AND GADUSOL SUNSCREEN FORMULATION

| | | |
|---|---|---|
| Trade Name | INCI | SUPPLIER |

PHASE A

| | | | |
|---|---|---|---|
| Gadusol | | | 0.5 |
| Dermofeel PA-3 | Sodium Phytate, Aqua, | Evonik | 0.2 |
| | Alcohol | | |
| Rheocare XGN | Xanthan Gum | BASF | 0.3 |
| Natpure Cellgum | Microcrystalline Cellulose, | Sensient Beauty | 1.5 |
| Plus | Cellulose Gum | | |
| Glycerin | Glycerin | Various | 2.0 |
| Zemea Propanediol | Propanediol | DuPont Tate & Lyle Bio | 3.0 |
| | | Products | |
| Lincoserve CG | Caprylyl Glycol | Lincoln Manufacturing | 1.0 |

PHASE B

| | | | |
|---|---|---|---|
| Lecinol S-10 | Hydrogenated lecithin | Barnet Products | 0.4 |
| Montanov 82 | Cetearyl Alcohol, Coco- | Seppic | 4.0 |
| | Glucoside | | |
| Montanov S | Coco-Glucoside, Coconut | Seppic | 1.5 |
| | Alcohol | | |
| Crodacol CS50 | Cetearyl Alcohol | Croda Inc | 2.0 |
| Hectorite Gel Soft | Caprylic/Capric | MakingCosmetics Inc | 2.5 |
| | Triglyceride, | | |
| | Stearalkonium Hectorite, | | |
| | Propylene | | |
| | Carbonate | | |
| Zano 20 | Zinc Oxide (nano sized) | EverCare | 15.0 |
| Lexfeel Natural | Heptyl Undecylenate | Index | 5.0 |
| Lexfeel N5 MB | Diheptyl Succinate, | Index | 7.0 |
| | Capryloyl Glycerin/Sebacic | | |
| | Acid Copolymer | | |

PHASE C

| | | |
|---|---|---|
| 20% (w/v, aq) Citric | Citric Acid | adjust to pH 7 |
| Acid | | |

### Example 4: In Vitro Testing OG Whitening Of ZnO-Containing Sunscreens with and without Gadusol

Variations of the formulation in Table 3A were prepared to test whitening of the formulations for comparable SPF with and without added. The procedure used to examine whitening was as follows.

In vitro whitening testing using spectrophotometric measurements is detailed below.

A known amount of a test formulation being evaluated for whitening was placed in the center of a 75 mm×25 mm unfrosted, glass microscope slide.

The treated slide was placed on black sheet of paper and a second microscope slide was placed over the first by slowly lowering the second slide over the first starting from a first side and angling the second slide over the first.

A known weight (for example, a 25 g calibration weight, was placed on the center of the top slide. This was done to create consistent results between slides for each sample tested.

A White Calibration Cap was used to calibrate a Spectrophotometer CM-600d (Konica Minolta)

Measurements of the slide was conducted by placing the spectrophotometer on top of the glass slide and absolute values (hue: h, a*, b* and lightness: L*) of two non-overlapping points/targets from each prepared sample slide were recorded.

Optionally, using the White Calibration Cap as the target color, use ‘Difference’ to display the color difference from the target color.

3 samples per formula were evaluated for averaging of the results.

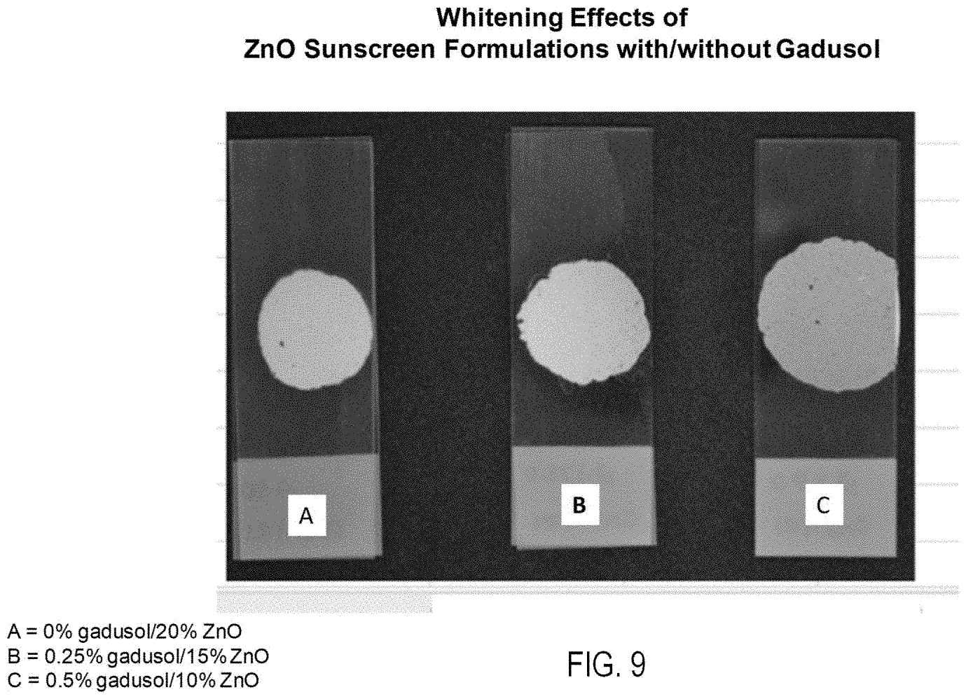

The following Table 4A and accompanying FIGS. 8-9, shows that the additional SPF imparted by gadusol provides a lesser whitening as measured using spectrophotometric testing sunscreen formulation while still providing excellent SPF. Moreover, since the ZnO used in this test was nano-sized, it could be expected that the lessening of whitening would be even more significant with use of a non-nano ZnO, such as that used in the COS 1 formulation in the previous examples.

TABLE 4A
WHITENING TESTS FOR VARIOUS SUNSCREEN FORMULATIONS

| Formulation | SPF | SD | L* |
|---|---|---|---|
| 0% G, 20% ZnO | 15.6 | 2.6 | 74.65 |
| 0.25% G, 15% ZnO | 29.0 | 2.6 | 70.63 |
| 0.5% G, 10% ZnO | 26.7 | 6.0 | 60.78 |

### Example 5: In Vivo/Human Subject Testing Whitening of ZnO-Containing Sunscreens with and without Gadusol

The formulations from Example 3 were tested to analyze the amount of residual whitening on human subjects having different Fitzpatrick Scale skin tones. The following procedure was used.

Determine and note subject's Fitzpatrick Phototype

Set up the portable photo studio light box and Nikon D3100 DSLR Camera on a tripod approximately 12 inches from the image target
Designate image target with a taped X or similar.

Outline an application site of approximately 30 cm² on the forearm of the volunteer.
Mark area

Clean and dry the test area with a dry cotton pad

Determine amount of test product to be applied (200 mg.cm²+/−2.5%)

Deposit droplets of the product using a syringe or pipette (approximately 15 droplets per 30 cm²), then spread the material over the test site with light pressure. Spreading time should be between 20-50 seconds depending upon the ease of spreading the product and site surface.

Allow to dry for approximately 5 minutes

Image volunteers arm in the light box (2) with the center of the application site placed on the image target designation.

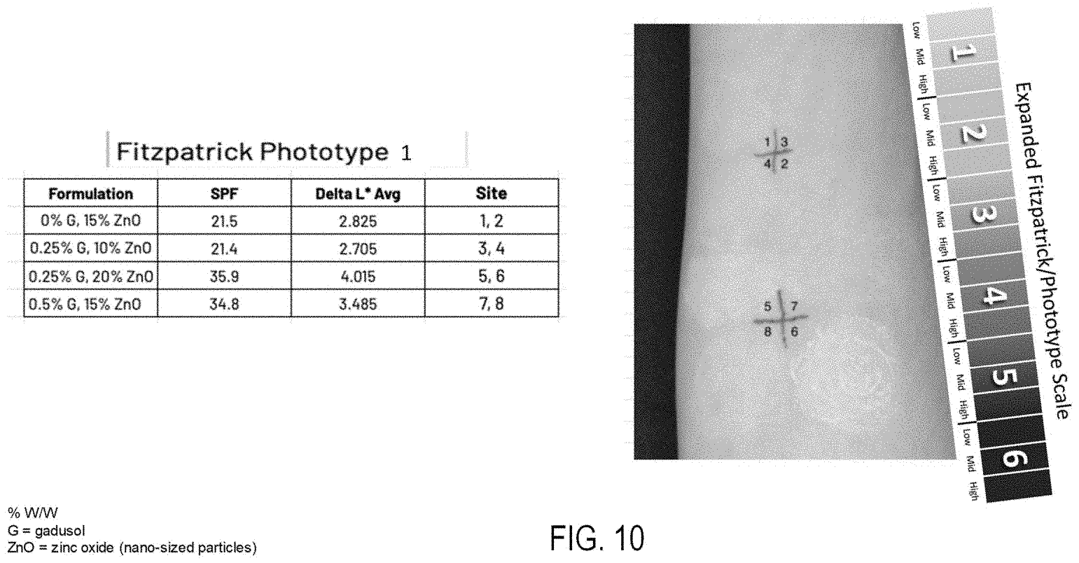

FIGS. 10-12 and the accompanying data tables demonstrate that on human subjects having Fitzpatrick Phototypes of 1, 4 and 5 there is a detectable L* decrease in whitening without reducing the in vitro SPF, by decreasing % ZnO and increasing % gadusol. The difference is visibly noticeable even on subject with a Fitzpatrick Phototype of 1 (see FIG. 10). This testing demonstrates that compositions comprising gadusol and ZnO can exhibit minimal residual whitening on human subjects with some Fitzpatrick Phototypes.

### Example 6: 10% ZnO, Gadusol and Porphyra-334 Composition

An oil-in-water sunscreen formulation comprising 10% ZnO (non-nano) and containing 2% gadusol and 1% porphrya-334 was prepared according to the ingredient listing below.

TABLE 6A
10% ZnO, GADUSOL AND PORPHYRA-334 COMPOSITION

| Ingredients: | % (w/w) |
|---|---|
| Water (Aqua) | 52.24 |
| Sodium Phytate | 0.05 |
| Xanthan Gum | 0.15 |
| Microcrystalline Cellulose; Xellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 4.00 |
| Coco-Glucoside; Coconut Alcohol | 1.50 |
| Cetearyl Alcohol | 1.00 |
| Behenyl Alcohol | 0.25 |
| Zinc Oxide; Caprylic/Capric Triglyceride; | 17.50 |
| Polyhydroxystearic Acid; Isostearic Acid (of | |
| which Zinc Oxide is 10 w/w %) | |
| Heptyl Undecylenate | 1.50 |
| Diheptyl Succinate; Caprylol Glycerin/Sebacid Acid | 9.00 |
| Copolymer | |
| Sorbitan Oleate | 0.10 |
| Silica | 1.00 |
| Citric Acid | <0.01 |
| Gadusol | 2 |
| Porphyra-334 | 1 |

In vitro SPF testing of this formulation resulted in a SPF of 40.1. This same formulation without gadusol or porphyra-334, resulted in an SPF of 16.6, thus indicating that the presence of 2% gadusol and 1% porphyra-334 increased SPF activity by 23.5 units. The UVA/UVB ratio was 0.68.

### Example 7: 4.5% ZnO, Gadusol and Tinosorb

In another example, an oil-in-water sunscreen formulation contains 0.5% gadusol, 4.5% zinc oxide, and a total of 10% Tinosorb M and/or Tinosorb S (w/w), follows.

TABLE 7A
COS 3 FORMULATION INCLUDING ZnO, GADUSOL, AND TINOSORB

| | INGREDIENT | | | |
|---|---|---|---|---|
| | (TRADE | | | % BY |
| PHASE | NAME) | INCI DESIGNATION | SUPPLIER | WEIGHT |
| A | WATER | WATER (AQUA) | LOCAL | 41.25 |
| A | DERMOFEEL | SODIUM PHYTATE | DR. | 0.05 |
| | PA-12 | | STRAETMANS/ | |
| | | | KINETIK | |
| A | KELTROL CG- | XANTHAN GUM | CPKELCONMCD | 0.15 |
| | SFT | | US | |
| A | AVICEL PC 611 | MICROCRYSTALLINE | FMC/EARTH | 1.50 |
| | | CELLULOSE | SUPPLIED PROD. | |
| | | CELLULOSE GUM | | |
| A | GLYCERIN | GLYCERIN | DEWOLF/AZELIS | 1.00 |
| | 99.7% USP- | | | |
| | AZELIS | | | |
| A1 | ZEMEA | PROPANEDIOL | DUPONT/ESSENTIAL | 3.00 |
| | PROPANEDIOL | | ING. | |
| A1 | SENSIVA SC-50 | ETHYLHEXYLGLYCERIN | SCHULKE & | 1.00 |
| | | | MAYR | |
| A1 | SENSIVA SC-10 | CAPRYLYL GLYCOL | SCHULKE & | 1.00 |
| | | ETHYLHEXYLGLYCERIN | MAYR | |
| B | LECINOL S-10 | HYDROGENATED | BARNIET | 0.20 |
| | | LECITHIN | | |
| B | MONTANOV 82 | CETEARYL ALCOHOL | SEPPIC | 4.00 |
| | | COCO-GLUCOSIDE | | |
| B | MONTANOV S | COCO-GLUCOSIDE | SEPPIC | 1.50 |
| | | COCONUT ALCOHOL | | |
| B | SP CRODACOL | CETEARYL ALCOHOL | CRODA | 1.00 |
| | C550 MBAL- | | | |
| | PA-(MH) | | | |
| B | LANETTE 22 | BEHENYL ALCOHOL | BASF/DEWOLF | 0.25 |
| B | LIPONATE GC | CAPRYLIC/CAPRIC | VANTAGE | 7.50 |
| | | TRIGLYCERIDE | (FORMERLY LIPO) | |
| B | LEXFEEL | HEPTYL UNDECYLENATE | INOLEX | 1.50 |
| | NATURAL | | | |
| B | LEXFEEL N5 | DIHEPTYL SUCCINATE | INOLEX | 7.00 |
| | | CAPRYLOYL | | |
| | | GLYCERIN/SEBACIC ACID | | |
| | | COPOLYMER | | |
| B | LEXFEEL N100 | DIHEPTYL SUCCINATE | INOLEX | 2.00 |
| | | CAPRYLOYL | | |
| | | GLYCERIN/SEBACIC ACID | | |
| | | COPOLYMER | | |
| B | SPAN 80-LQ- | SORBITAN OLEATE | CRODA | 0.10 |
| | (AP) | | | |
| B | MSS-500W | SILICA | KOBO | 0.50 |
| B | MSS-5003H | SILICA | KOBO | 0.50 |
| C | TINOSORB M | METHYLENE BIS- | BASF/DEWOLF/ | 20.00 |
| | | BENZOTRIAZOLYL | AZELIS | |
| | | TETRAMETHYLBUTYLPHENOL | | |
| | | (BISOCTRIZOLE) | | |
| | | WATER (AQUA) | | |
| | | DECYL GLUCOSIDE | | |
| | | PROPYLENE GLYCOL | | |
| | | XANTHAN GUM | | |
| D | CITRIC ACID | WATER CITRIC ACID | SPECTRUM | 0.00 |
| | USP 20% SOLN | | | |

Instructions for preparing COS 3 were similar to that of COS 1 and COS 2 above. Also, as with the COS 1 and COS 2 formulations, the amount of ZnO and gadusol was varied in the formulations to see the various effects of ingredients on the resulting SPF. The ZnO was of the non-nano type (Alfa Aesar 200 (72 micron) mesh powder).

Table 7B below shows the increase in in vitro SPF as a result of gadusol addition. This demonstrates the improvement in SPF with Tinosorb and 10% ZnO.

TABLE 7B
IN VITRO SPF RESULTS FOR ZnO, GADUSOL,
AND TINOSORB

| Base | For- | | Gadusol | Tinosorb | | Gadusol SPF |
|---|---|---|---|---|---|---|
| For- | mulation | ZnO | (w/w) | M | In | Boosting |
| mulation | Code | % | % | (w/w) % | vitro SPF | Effect |
| Cos3 | Glabs27 | 0 | 0 | 10 | 26.55 ± 3.1 | N/A |
| | | | | | | (Control) |
| Cos3 | Glabs28 | 0 | 0.5 | 10 | 46.8 ± 6.9 | −20.25 |
| Cos3 | Glabs29 | 4.5 | 0 | 10 | 41.3 ± 7 | N/A |
| | | | | | | (Control) |
| Cos3 | Glabs30 | 4.5 | 0.5 | 10 | 71.1 ± 9 | −29.8 |

### Example 8: Antioxidant Activity of Gadusol Compared to Porhyra-334

The antioxidant capacity of gadusol and porphyra-334 were tested using the well-known Folin-Ciocalteu method to generate a dose response curve indicating that gadusol at concentrations ranging from about 0.3 to 10 mg/ml has the antioxidant power substantially equivalent to a mycosporine compound from “nori” seaweed. The results show that, when combined, the two compounds exhibit UVA/UVB photo protective activity over a larger spectrum than each does individually. The cell-based antioxidant protection in erythrocytes (CAP-e) assay (Honzel et. al., 2008, and Jensen GS. Cell-based antioxidant protection assay, also disclosed in U.S. Pat. No. 8,465,988, incorporated herein in its entirety by this reference) was used. This testing was selected to measure antioxidant power but, in addition, the method ensures that the antioxidant agent crosses the cell's lipid boundary layer so that only intracytoplasmic activity is measured. At the highest dose (3.3 mg/ml), both gadusol and porphyra-334 caused some lysis of the cells, which precluded testing of higher concentrations. However, at about 1 mg/ml, gadusol was a significantly more active antioxidant in vivo than the porphyra-334. Where the two were combined, the porphyra-334 did not interfere with the protective action of the gadusol. Some in vivo antioxidant activity was seen for gadusol even at 0.133 mg/ml. The tested concentrations for both gadusol and the porphyra-334 ranged from 0.001 to 3.33 mg/mL.

### Example 9: 5% ZnO, Gadusol and Polysolicone-15 (Prophetic)

In another example, an oil-in-water sunscreen formulation can contain 0.5% gadusol, 5% zinc oxide (non-nano), and a total of 4% polysilicone-15 (w/w).

TABLE 9A
PROPHETIC COMPOSITION ZnO, GADUSOL AND POLYSOLICONE-15

| Ingredients: | % (w/w) |
|---|---|
| Water (Aqua) | 50.74 |
| Sodium Phytate | 0.05 |
| Xanthan Gum | 0.15 |
| Microcrystalline Cellulose; Cellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 4.00 |
| Coco-Glucoside; Coconut Alcohol | 1.50 |
| Cetearyl Alcohol | 1.00 |
| Behenyl Alcohol | 0.25 |
| Zinc Oxide; Caprylic/Capric Triglyceeride; | 8.75 |
| Polyhydroxystearic Acid; | |
| lsostearic Acid (of which Zinc Oxide is 5% w/w) | |
| Heptyl Undecylenate | 1.50 |
| Diheptyl Succinate; Caprylol Glycerin/Sebacid Acid Copolymer | 9.00 |
| Sorbitan Oleate | 0.10 |
| Silica | 1.00 |
| Citric Acid | <0.01 |
| Gadusol | 0.5 |
| Polysilicone-15 | 4 |

### Example 10: 2% ZnO and Gadusol (Prophetic)

Yet another example formulation for a topical that included gadusol is shown below.

TABLE 10A
PROPHETIC COMPOSITION 2% ZnO AND GADUSOL

| Ingredient | Function | Formula 1 |
|---|---|---|
| petrolatum | base | 3.0 |
| Coconut oil | moisturizer | 3.0 |
| Zinc oxide | UV blocker | 2.0 |
| Gadusol | UV filter | 0.5 to 2.0 |
| Tween-80 | Emulsifier | 1.0 |
| Carbapol-940 | Thickener | 1.0 |
| Triethanolamine | Buffering agent | To pH 7.0 |
| Water | QS to 100 gm | |

### Example 11: TiO_{2,} Gadusol, Gadusporine, and Scytonemin Sunscreen Formulation (Prophetic)

Yet another example formulation for a topical that includes gadusol is shown below.

TABLE 11A
PROPHETIC COMPOSITION OF TiO2, GADUSOL, GADUSPORINE,
AND SCYTONEMIN SUNSCREEN FORMULATION

| | |
|---|---|
| Titanium dioxide | 5 |
| Zinc oxide | 5 |
| Gadusol | 5-10 |
| Scytonemin | 2 |
| Gadusporine-A* | 0.1 |
| Olive oil | 1 |
| Sorbitan monooleate | 0.5 |
| Glycerol tricaprylate | 1 |
| Caprylic acid | 15 |
| Glycerin | 3 |
| Magnesium sulfate | 0.1 |
| Butylene glycol | 5 |
| Purified water | QS to 100 grams, adjust pH to 6.2 |

*A mix of MAA or gadusporines could substitute.

### Example 12 Gadusol and Porphyra-334 (Prophetic)

Yet another example formulation for a topical that included gadusol and porphyra-334 is shown below.

TABLE 12A
PROPHETIC COMPOSITION GADUSOL AND PORPHYRA-334

| | |
|---|---|
| Neo-PCl (Symrise) | 5 g (trideceth-9, PEG-5- |
| | ethylhexanoate and |
| | water) as hydrating |
| | emollient |
| Propylene glycol | 1.25 ml |
| Gadusol | 1.0 ml (as a 10% |
| | bufferedaqueous |
| | solution) |
| Porphyra-334 | 0.75 ml (as a 10% |
| | buffered aqueous |
| | solution) |

### Example 13: Gadusol and MAA's/Gadusporine (Prophetic)

Yet another example formulation for a topical that included gadusol along with MAA's or gadusporines is shown below.

TABLE 13A
PROPHETIC COMPOSITION OF GADUSOL AND
MMA’S/GADUSPORINE

| | | For- mula | For- mula | For- mula |
|---|---|---|---|---|
| Ingredient | Function | 1 | 2 | 3 |
| Caprylate/capric | Emollient | 1.6 | 1.6 | 1.6 |
| triglycerides | | | | |
| lsopropyl myristate | Permeation enhancer | 0 | 5.0 | 0 |
| Dimethylisosorbide | Permeation enhancer | 0 | 0 | 5.0 |
| Beeswax | Lubricant | 1.0 | 1.0 | 1.0 |
| Cetyl palmitate | Emollient/stability | 0.5 | 0.5 | 0.5 |
| | enhancer | | | |
| Glutamyl-N-palmitate | Emulsifier | 0.6 | 0.6 | 0.6 |
| (Amisoft ®) | | | | |
| EDTA | Chelating agent | 0.01 | 0.01 | 0.01 |
| Glycerin | Humectant | 3.0 | 3.0 | 3.0 |
| Gadusol | UV Photoprotectant | 0.5-10.0 | 0.5-10.0 | 0.5-10.0 |
| Porphyra-334 or any | UV Photo protectant | 5.0 | 5.0 | 5.0 |
| MAA or mixture of MAA | | | | |
| or gaduporines | | | | |
| Phenoxyethanol | Preservative | 0.2 | 0.2 | 0.5 |
| Triethanolamine | | To | To | To |
| | | pH 6.5 | pH 6.5 | pH 6.5 |
| Water | QS to 100 gm | | | |

### Example 14: Gadusol with MAA's/Gadusporines/Scytonemin (Prophetic)

Another example includes the formulation including scytomenin (as an additional UV absorber) detailed below.

TABLE 14A
PROPHETIC COMPOSITION WITH GADUSOL WITH MAA’S,
GADUSPORINES, AND SCYTONEMIN

| | | Formula |
|---|---|---|
| Ingredient | Function | 1 |
| Mineral oil | Emollient | 13.0 |
| Squalane oil | moisturizer | 3.0 |
| Glyceryl monostearate | emulsifier | 1.0 |
| Sorbitan monostearate | Emulsifier/wetting agent | 1.0 |
| EDTA | Chelating agent | 0.01 |
| Glycerin | Humectant | 5.0 |
| Gadusol | UV Photo protectant | 0.5-10.0 |
| Gadusporine A or mix of | UV Photo protectant | 5.0 |
| gadusporines or MAA | | |
| Scytonemin | UV Photo protectant | 5.0 |
| Phenoxyethanol | Preservative | 0.5 |
| Triethanolamine | | To pH 6.5 |
| Water | QS to 100 gm | |

### Example 15: Gadusol, Gadusporine A Composition (Prophetic)

Another example includes the formulation detailed below, composition is given as % (w/w). In a further example, a composition could be formulated and ingredients adjusted specifically for sun protection or other indications, e.g., antioxidant activity. For example, an embodiment includes the formulation detailed below, % (w/w).

TABLE 15A
PROPHETIC COMPOSITION OF GADUSOL, GADUSPORINE A
COMPOSITION

| | |
|---|---|
| Polysorbates or polyglyceryl oleate emulsifier | 2-10% |
| Potassium L-Glutamyl-N-(1-oxohexadecyl) (CAS#111391-27-6) | 1-4% |
| L-alanine-N-(1-oxohexadecyl) | 1-4% |
| Emollients 3-10% short chain acetyl triglycerides | 5-10% |
| Grapeseed oil | 5-20% |
| Stearyl palmitate, cetyl alcohol thickener | 0-2% |
| Gadusol | 0.5-10% |
| Gadusporine A* | 1-2% |
| Glycerin, or propylene glycol Humectant | 2-5% |
| Teatree essential oil as a preservative | 0.1-1.0% |
| Water | 50-70% |

*A mix of MAA or gadusporines could substitute

### Example 16: Gadusol-Containing Cosmeceutical Formulation (Prophetic)

Another example formulation could be prepared including the formulation detailed below (e.g., prepared as a sunscreen formulation). Such a formulation could have utility as an antioxidant cosmeceutical skincare product.

Starting with 12 gm of USP (pharmaceutical) water at 30 C, add:

3 gm Gadusol

3 gm Gadusporine A*

0.8 gm Tocopheryl succinate (free acid) as lipophilic ion pair with Gadusporine 1 gm dodexyl 4,6 diene tyrosine fatty acid amide

1 gm PEG-300
*A mix of MAA or gadusporines could substitute

### Example 17: Gadusol Containing Formulation with Antioxidant Ingredients (Prophetic)

Another possible composition that can be useful as a cosmeceutical formulation, could be prepared as follows:

Starting with 20 gm of USP water at 30 C, add:

3 gm Gadusol

3 gm Gadusporine A*

0.8 gm Tocopheryl succinate (free acid) as lipophilic ion pair with Gadusporine

1 gm PEG-300

0.1 gm retinoic acid (active) in 200 ul USP ethanol 1 gm b-carotene to form an oil-in-water emulsion 10 mg disodium EDTA

0.05 gm phenoxyethanol as a preservative Adjust pH with TEA/succinic acid to pH 6.6

*A mix of MAA or gadusporines could substitute

### Example 18: Gadusol-Containing Formulation with Plant Oil Emollients (Prophetic)

Another possible composition that can be useful as a cosmeceutical formulation, could be prepared as follows:

TABLE 18A
PROPHETIC COMPOSITION OF GADUSOL-CONTAINING
FORMULATION WITH PLANT OIL EMOLLIENTS

| | |
|---|---|
| Polysorbate emulsifier | 2-6% |
| Glyceryl palmitate | 0.1-2% |
| Plant oil emollients | 10-35% |
| Gadusol | 2-10% |
| Short chain triglycerides | 5% |
| Benzyl alcohol-DHA | 0.1-1.0% |
| Water | 60-80% |
| Fragrance to suit | |

### Example 19: Gadusol UVB Composition (Prophetic)

Gadusol could also be used at >0.5 to 10% as a standalone UVB-protective sunscreen.

TABLE 19A
PROPHETIC COMPOSITION GADUSOL UVB COMPOSITION
(PROPHETIC)

| INCI Name | % (w/w) |
|---|---|
| Water (Aqua) | 62.25 |
| Sodium Phytate | 0.05 |
| Xanthan Gum | 0.15 |
| Microcrystalline Cellulose; Cellulose Gum | 1.50 |
| Glycerin | 1.00 |
| Propanediol | 3.00 |
| Ethylhexylglycerin | 1.00 |
| Capylyl Glycol; Ethylhexylglycerin | 1.00 |
| Hydrogenated Lecithin | 0.20 |
| Cetearyl Alcohol; Coco-Glucoside | 9.00 |
| Coco-Glucoside; Coconut Alcohol | 1.50 |
| Cetearyl Alcohol | 1.00 |
| Behenyl Alcohol | 0.25 |
| Heptyl Undecylenate | 1.50 |
| Diheptyl Succinate; Caprylol Glycerin/Sebacid Acid Copolymer | 14.00 |
| Sorbitan Oleate | 0.10 |
| Silica | 1.00 |
| Citric Acid | <0.01 |
| Gadusol | 0.5-10% |

The disclosure also provides support for a sunscreen formulation, comprising: a physical/inorganic sunscreen active ingredient at from about 5% to about 25% on a weight/weight (w/w) basis, wherein the active ingredient generates a first sun protection factor (“SPF”) value to a sunscreen formulation, gadusol, and a plurality of ingredients configured to deliver ingredients a) and b) in the sunscreen formulation, wherein the gadusol is present in the sunscreen formulation in an amount sufficient to generate a SPF booster effect in the sunscreen formulation. In a first example of the sunscreen formulation, the booster effect of the gadusol generates an SPF value for the sunscreen formulation of at least 1.5 times the first SPF value generated by the physical/inorganic sunscreen active ingredient when the active ingredient is present at a same amount in a formulation that does not contain gadusol. In a second example of the sunscreen formulation, optionally including the first example having an SPF of at least about 25. In a third example of the sunscreen formulation, optionally including one or both of the first and second examples, the physical/inorganic sunscreen active ingredient is ZnO. In a fourth example of the sunscreen formulation, optionally including one or more or each of the first through third examples, the ZnO has an average particle size of greater than about 100 nm. In a fifth example of the sunscreen formulation, optionally including one or more or each of the first through fourth examples, the ZnO has an average particle size of less than about 100 nm. In a sixth example of the sunscreen formulation, optionally including one or more or each of the first through fifth examples, the amount of gadusol is from about 0.25 to about 1.0%. In a seventh example of the sunscreen formulation, optionally including one or more or each of the first through sixth examples, the physical/inorganic sunscreen active ingredient is present at about 15% or less. In an eighth example of the sunscreen formulation, optionally including one or more or each of the first through seventh examples, the first SPF value is determined using an in vivo method. In a ninth example of the sunscreen formulation, optionally including one or more or each of the first through eighth examples, the first SPF value is determined using an in vitro method. In a tenth example of the sunscreen formulation, optionally including one or more or each of the first through ninth examples, the sunscreen formulation generates minimal residual whitening effect when applied to a skin surface of a person in need of protection from UV radiation. In an eleventh example of the sunscreen formulation, optionally including one or more or each of the first through tenth examples, the sunscreen formulation further comprises: gadusporine at from about 0.25 to about 1.0% (w/w) of the sunscreen formulation. In a twelfth example of the sunscreen formulation, optionally including one or more or each of the first through eleventh examples, the sunscreen formulation further comprises: a mycosporine-like amino acid at from about 0.25 to about 1.0% (w/w) of the sunscreen formulation.

The disclosure also provides support for a method of making a sunscreen formulation comprising the steps of: providing from about 0.25% to about 1.0% gadusol on a weight/weight (w/w) basis, providing from about 5 to about 25% (w/w) of a physical/inorganic sunscreen active ingredient, providing a plurality of ingredients configured to generate a delivery system for the gadusol and the physical/inorganic sunscreen active ingredient, and preparing a sunscreen formulation comprising the gadusol and the physical/inorganic sunscreen active ingredient, wherein the sunscreen formulation: does not incorporate a chemical/organic sunscreen active ingredient, and has a sun protection factor (SPF) of at least about 15. In a first example of the method, the chemical/organic sunscreen active ingredient not incorporated in the sunscreen formulation comprises each of: cinoxate, dioxybenzone, ensulizole, homosalate, meradimate, octinoxate, octisalate, octocrylene, padimate 0, sulisobenzone, oxybenzone, and avobenzone. In a second example of the method, optionally including the first example, the SPF is at least about 25. In a third example of the method, optionally including one or both of the first and second examples, the physical/inorganic sunscreen active ingredient comprises zinc oxide. In a fourth example of the method, optionally including one or more or each of the first through third examples, the sunscreen formulation generates minimal residual whitening when applied to a skin surface of a user in need of protection from UV radiation. In a fifth example of the method, optionally including one or more or each of the first through fourth examples, the physical/inorganic sunscreen active ingredient has an average particle size of greater than 100 nm. In a sixth example of the method, optionally including one or more or each of the first through fifth examples, the method further comprises: providing gadusporine at from about 0.25 to about 1.0% (w/w) of the sunscreen formulation.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A sunscreen formulation, comprising:
a) a physical/inorganic sunscreen active ingredient at from about 5% to about 25% on a weight/weight (w/w) basis, wherein the active ingredient generates a first sun protection factor (“SPF”) value to a sunscreen formulation;
b) gadusol; and
c) a plurality of ingredients configured to deliver ingredients a) and b) in the sunscreen formulation,
wherein the gadusol is present in the sunscreen formulation in an amount sufficient to generate a SPF booster effect in the sunscreen formulation.

2. The sunscreen formulation of claim 1, wherein the booster effect of the gadusol generates an SPF value for the sunscreen formulation of at least 1.5 times the first SPF value generated by the physical/inorganic sunscreen active ingredient when the active ingredient is present at a same amount in a formulation that does not contain gadusol.

3. The sunscreen formulation of claim 2 having an SPF of at least about 25.

4. The sunscreen formulation of claim 1, wherein the physical/inorganic sunscreen active ingredient is ZnO.

5. The sunscreen formulation of claim 4, wherein the ZnO has an average particle size of greater than about 100 nm.

6. The sunscreen formulation of claim 4, wherein the ZnO has an average particle size of less than about 100 nm.

7. The sunscreen formulation of claim 1, wherein the amount of gadusol is from about 0.25 to about 0%.

8. The sunscreen formulation of claim 1, wherein the physical/inorganic sunscreen active ingredient is present at about 15% or less.

9. The sunscreen formulation of claim 1, wherein the first SPF value is determined using an in vivo method.

10. The sunscreen formulation of claim 1, wherein the first SPF value is determined using an in vitro method.

11. The sunscreen formulation of claim 1, wherein the sunscreen formulation generates minimal residual whitening effect when applied to a skin surface of a person in need of protection from UV radiation.

12. The sunscreen formulation of claim 1, further comprising gadusporine at from about 0.25 to about 1.0% (w/w) of the sunscreen formulation.

13. The sunscreen formulation of claim 1, further comprising a mycosporine-like amino acid at from about 0.25 to about 1.0% (w/w) of the sunscreen formulation.

14. A method of making a sunscreen formulation comprising the steps of:
a) providing from about 0.25% to about 1.0% gadusol on a weight/weight (w/w) basis;
b) providing from about 5 to about 25% (w/w) of a physical/inorganic sunscreen active ingredient;
c) providing a plurality of ingredients configured to generate a delivery system for the gadusol and the physical/inorganic sunscreen active ingredient; and
d) preparing a sunscreen formulation comprising the gadusol and the physical/inorganic sunscreen active ingredient,
wherein the sunscreen formulation:
i) does not incorporate a chemical/organic sunscreen active ingredient; and
ii) has a sun protection factor (SPF) of at least about 15.

15. The method of claim 14, wherein the chemical/organic sunscreen active ingredient not incorporated in the sunscreen formulation comprises each of: cinoxate, dioxybenzone, ensulizole, homosalate, meradimate, octinoxate, octisalate, octocrylene, padimate O, sulisobenzone, oxybenzone, and avobenzone.

16. The method of claim 14, wherein the SPF is at least about 25.

17. The method of claim 14, wherein the physical/inorganic sunscreen active ingredient comprises zinc oxide.

18. The method of claim 14, wherein the sunscreen formulation generates minimal residual whitening when applied to a skin surface of a user in need of protection from UV radiation.

19. The method of claim 14, wherein the physical/inorganic sunscreen active ingredient has an average particle size of greater than 100 nm.

20. The method of claim 14, further comprising providing gadusporine at from about 0.25 to about 1.0% (w/w) of the sunscreen formulation.
